(19) United States
(12) Patent Application Publication
(10) Pub. No.: US 2006/0189893 A1
(43) Pub. Date: Aug. 24, 2006

(54) Systems and methods for detecting abnormal cells

(75) Inventors: Warren Maltzman, Novato, CA (US); Peter Gombrich, Novato, CA (US); Eugene Dimonte, Aurora, IL (US); Edward Eaton, Eola, IL (US); Eric Larson, Pecatonica, IL (US)

Correspondence Address: HAMRE, SCHUMANN, MUELLER & LARSON, P.C., P.O. BOX 2902-0902, MINNEAPOLIS, MN, 55402, US

(73) Assignee: DIAMICS, INC., Novato, CA (US)

(21) Appl. No.: 11/318,123

(22) Filed: Dec. 23, 2005

Related U.S. Application Data
(60) Provisional application No. 60/642,008, filed on Jan. 6, 2005.
(60) Provisional application No. 60/681,901, filed on May 17, 2005.
(60) Provisional application No. 60/686,150, filed on Jun. 1, 2005.
(60) Provisional application No. 60/708,150, filed on Aug. 15, 2005.
(60) Provisional application No. 60/729,854, filed on Oct. 25, 2005.
(60) Provisional application No. 60/729,857, filed on Oct. 25, 2005.

(51) Int. Cl. A61B 10/00

(52) U.S. Cl. 600/569; 600/562

(57) ABSTRACT

Systems and methods of interrogating clusters of cells for two or more biological markers that do not, or rarely, occur in the same cell during normal cellular growth, development and function, to indicate the existence of cells that are part of a local area where a pre-neoplastic or neoplastic lesion may be present. The relationship among cells is maintained while interrogating the clusters of cells to facilitate the examination and determination of the existence of possible dysplasia.

This application claims the benefit of U.S. Provisional Application No. 60/642,008 filed Jan. 6, 2005; U.S. Provisional Application No. 60/681,901 filed May 17, 2005; U.S. Provisional Application No. 60/686,150 filed Jun. 1, 2005; U.S. Provisional Application No. 60/708,150 filed Aug. 15, 2005; U.S. Provisional Application No. 60/729,854 filed Oct. 25, 2005; and U.S. Provisional Application No. 60/729,857 filed Oct. 25, 2005. Each of the above-referenced applications is incorporated herein by reference in their entirety.

### TECHNICAL FIELD

This disclosure relates generally to cell sampling and screening for use in detecting abnormal tissues in the body, for example in the cervix. More specifically, this disclosure relates to systems and methods whereby clusters of cells are collected in a manner where the spatial arrangement of the collected clusters of cells is preserved, and the biological properties of such clusters are examined with respect to the expression of two or more features.

### BACKGROUND

It is often necessary to collect various cell samples from patients for the purposes of screening for, detecting, and ultimate treatment of, a number of diseases and abnormalities. One of the major reasons for the collection of cellular samples is for the purpose of screening patients for cancer. For example, urine, sputum, breast nipple and fine needle aspirates, and exfoliated cells of the uterine cervix are screened by cytotechnicians and pathologists for the presence of abnormal cells suggestive of the presence of a solid tumor. When such suspicious cells are found, a more definitive diagnosis is reached by removing a sample of the tissue where a lesion is suspected, and submitting the sample for review by a pathologist.

A major issue with any screening test, or preliminary diagnostic test, is that it must be sensitive enough to detect disease, but specific enough not to classify unaffected individuals at such a high frequency as to present an emotional or physical burden. This is especially true for those screening tests, such as cervical cytology (commonly termed the Pap test), which are routinely applied to large populations without regard to a heightened index of suspicion of the presence of disease.

It is generally accepted that diagnosis of cancer at its earliest stages affords the greatest opportunity for effective treatment. A corollary to this is that early diagnosis of a solid tumor corresponds to recognition of localized abnormalities, which at the cellular level are not that different from the surrounding tissue. This presents a challenge for screening of cellular samples where all context and comparison to neighboring cells is lost. One approach to this problem is to concentrate upon elements, i.e. groups of cells, which more closely approximate intact tissue elements. In fact, the presence of such clusters of cells, in and of itself, can be considered to be suggestive of a pre-cancerous or cancerous condition. However, it is also the case that normal tissue elements can be represented as cell clusters in samples collected for cytologic analysis.

Preneoplastic lesions present unique biological features. Dysplasia, the early phase of neoplastic progression, involves cells that are individually minimally different from normal cells present in the same tissue. The major difference between a dysplastic lesion and normal tissue elements undergoing changes in shape (metaplasia) or actively proliferating (hyperplasia) is an imbalance in the fractions of cells expressing characteristic proteins involving abnormal cell growth and turnover. It is well recognized by pathologists, who examine intact tissues, that the admixture of morphological (e,g, mitotic figures) or biochemical (e.g. Ki-67 proliferation antigen) markers of normal growth and function with morphological (e.g. apoptotic bodies) or biochemical (e.g. activated caspase 3) indicators of cell turnover by the process of apoptosis, is characteristic of dysplasia.

Conventional sampling methods utilized in current screening procedures acquire cells from a lesion, but then disperse these cells into a typically much larger number of normal cells obtained from outside of the boundaries of the lesion. This dispersion results in the evaluation of a sample being an exercise in the detection of a rare event; that is, finding one or a few abnormal cells within a background consisting of a very large number (e.g. 50,000-300,000) of normal cells. Furthermore, and perhaps most significantly, dispersion eliminates the information that can be gained from determining the biological characteristics of small areas that might represent preneoplastic lesions. This essential information is present in the relationship among cells, and is not apparent by examining individual cells in isolation from adjacent cells within a tissue. Dispersion also precludes using the sample to determine the location of the lesion on the patient.

Therefore, it would also be desirable to incorporate the unique biological features of preneoplastic lesions with a means to collect and analyze clusters of cells, and screen cellular samples for the presence of cell clusters indicative of dysplasia in the sampled tissue.

### SUMMARY

The invention relates to systems and methods to screen cellular samples for the presence of cell clusters indicative of dysplasia in the sampled tissue. Clusters of cells are interrogated for two or more biological markers that do not, or rarely, occur in the same cell during normal cellular growth, development and function, to indicate the existence of cells that are part of a local area where a pre-neoplastic or neoplastic lesion (hereinafter “dysplasia”) may be present. The relationship among cells is maintained while interrogating the clusters of cells to facilitate the examination and determination of the existence of possible dysplasia of the tissue.

The concepts described herein can be implemented using biological markers that are not, or rarely, co-expressed in the same cell and the expression of which becomes imbalanced in dysplasia. The two or more markers that are screened can result from an imbalance in the fractions of cells expressing characteristic proteins involving abnormal cell growth and turnover. For example, the admixture of morphological (e,g, mitotic figures) or biochemical (e.g. Ki-67 proliferation antigen) markers of normal growth and function with morphological (e.g. apoptotic bodies) or biochemical (e.g. activated caspase 3) indicators of cell turnover by the process of apoptosis, is characteristic of dysplasia.

The concepts described herein can be used to screen for dysplasia in a number of regions of the body, for example from the cervix, the bladder, the lungs, the colon, the ovaries, and breasts. The clusters of cells can be analyzed as they naturally occur or they can be analyzed as they naturally occur or they can be collected from tissue, urine, induced sputum, breast secretions, cells washed from ovaries, and the like using a suitable collector.

The cell collector is preferably designed to enhance the capability of the collector to maintain the integrity of cellular clusters or clumps, and to facilitate transfer of the collected clusters of cells onto a receiving structure, for example a slide. In one embodiment, a combination of the material of the collector, the texture of the collection surface of the collector, and the use of expansion and rotation of the collector during collection facilitate the collection of the clusters of cells. Preferably, the collector can be expanded during transfer such that the cell clusters obtained from the endo- and ecto-cervical regions end up on a generally common plane for subsequent transfer to the receiving structure. Preferably, clusters of cells are transferred from the collector to the receiving structure in such a way as to retain the spatial relationships that existed between the cells in the clusters prior to sampling. Orientation marks on the collector and the receiving structure assist in maintaining the spatial relationship during transfer.

The collector is expanded during collection as well as during transfer of the cells. Expansion during collection and transfer can occur through the use of air, by a mechanical expansion system, or through a combination of air and a mechanical system.

### BRIEF DESCRIPTION OF FIGURES

FIGS. 1A-C illustrate the general features of a cervical analysis system utilizing the concepts of the present invention.

FIGS. 2A and 2B are a side view and a cross sectional view taken along line A-A, respectively, of one embodiment of a cell collector assembly according to the present invention.

FIG. 2C is a detailed view of the expandable collection tip of the cell collector assembly.

FIG. 3 is a cross-sectional view of the cell collector attached to a collector handle assembly.

FIG. 4 is a schematic diagram of a user's hand holding the cell collector attached to the collector handle assembly.

FIGS. 5A-C are cross sectional views of the tip region of the cell collector illustrating expansion of the cell collector tip during cell cluster collection.

FIGS. 6A-C illustrate the steps of cell cluster collection from a cervix using the cell collector.

FIGS. 7A-K illustrate the process of cell cluster transfer using the cell collector, with colored marker and marking fluid simulating collected clusters of cells.

FIG. 8 illustrates a cell transfer device on which the cell collector is mounted for transferring clusters of collected cells.

FIGS. 9A-C are views of a touch prep of cervical cells after labeling with markers.

FIG. 10 is a perspective view of a manual scanner device for use in analyzing collected clusters of cells.

FIG. 11 is a schematic view of an automatic scanner device for use in analyzing collected clusters of cells.

FIG. 12 illustrates another embodiment of a cell collector.

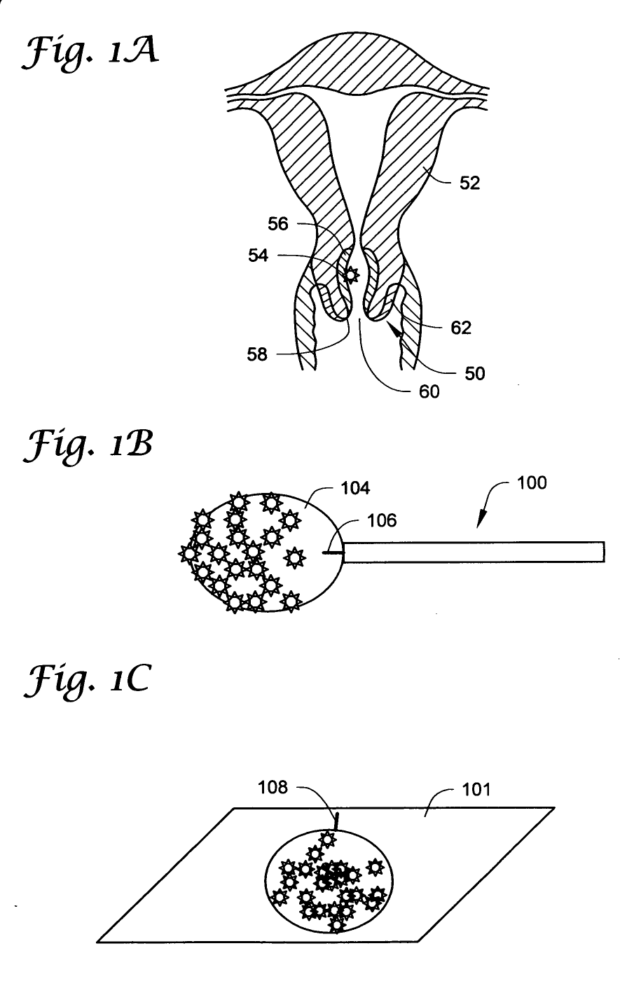

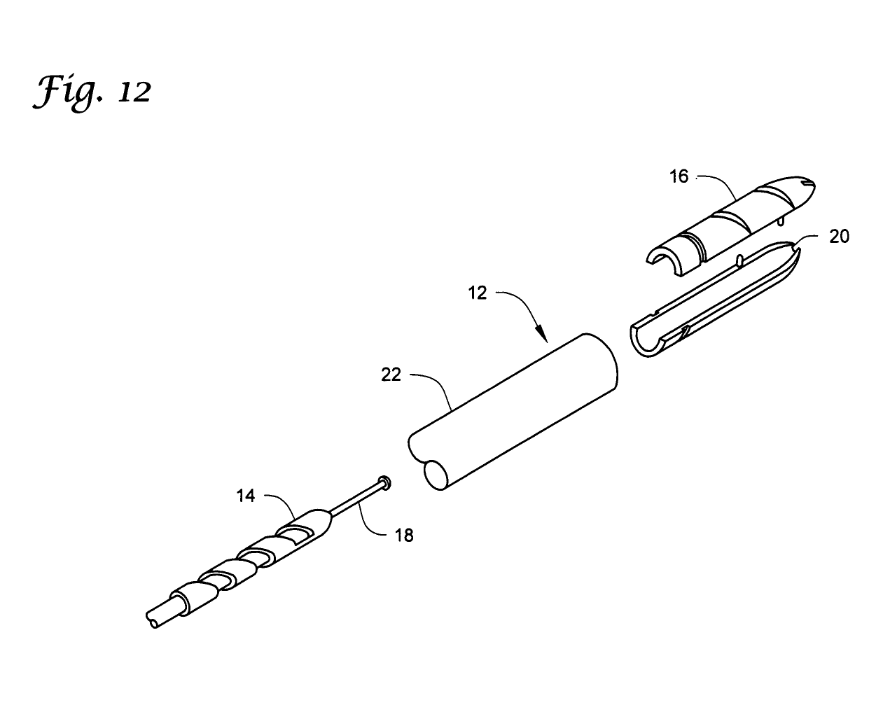

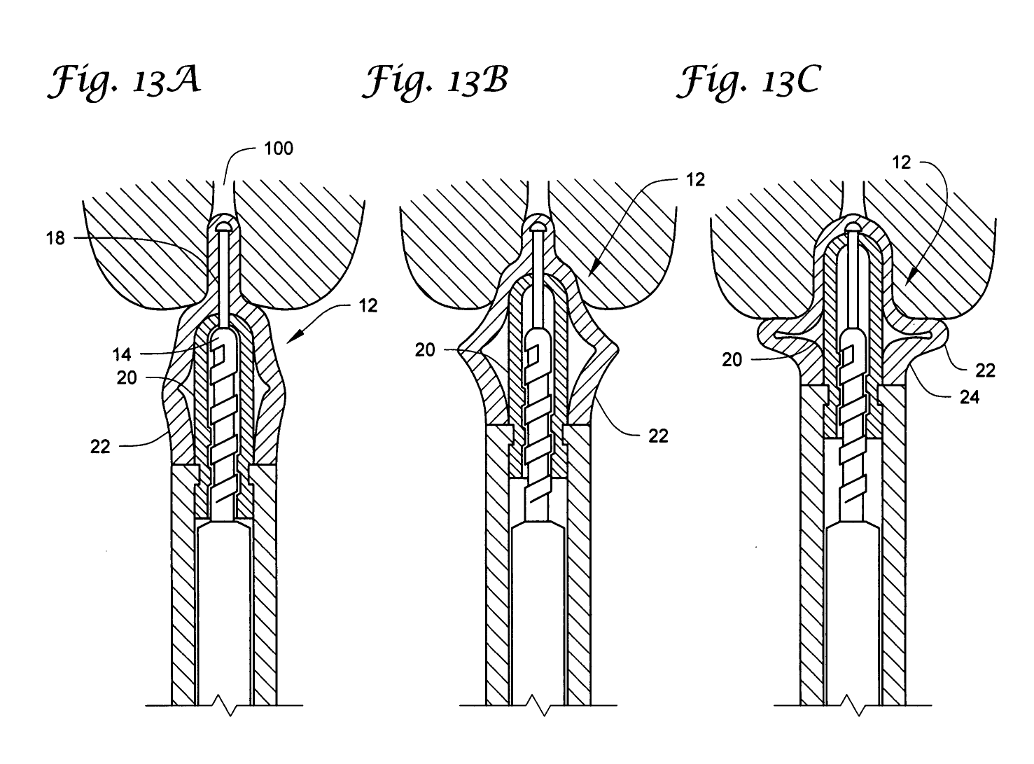

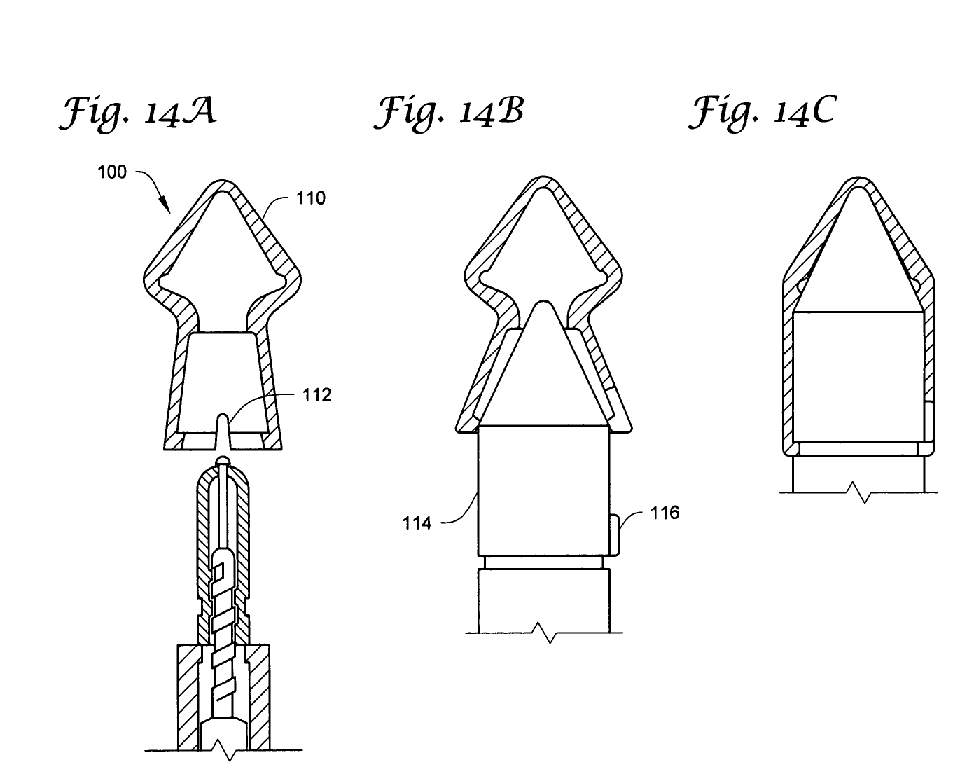

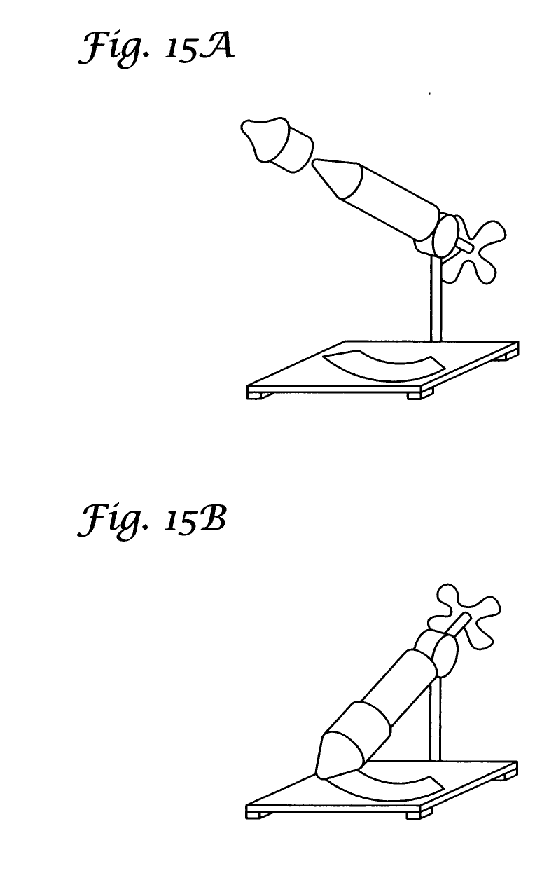

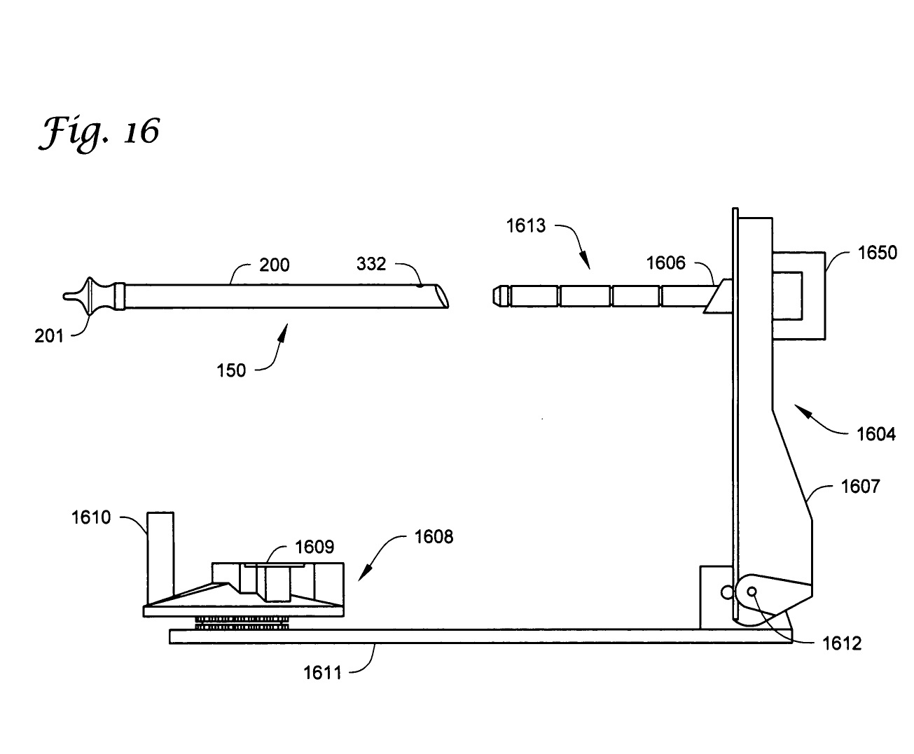

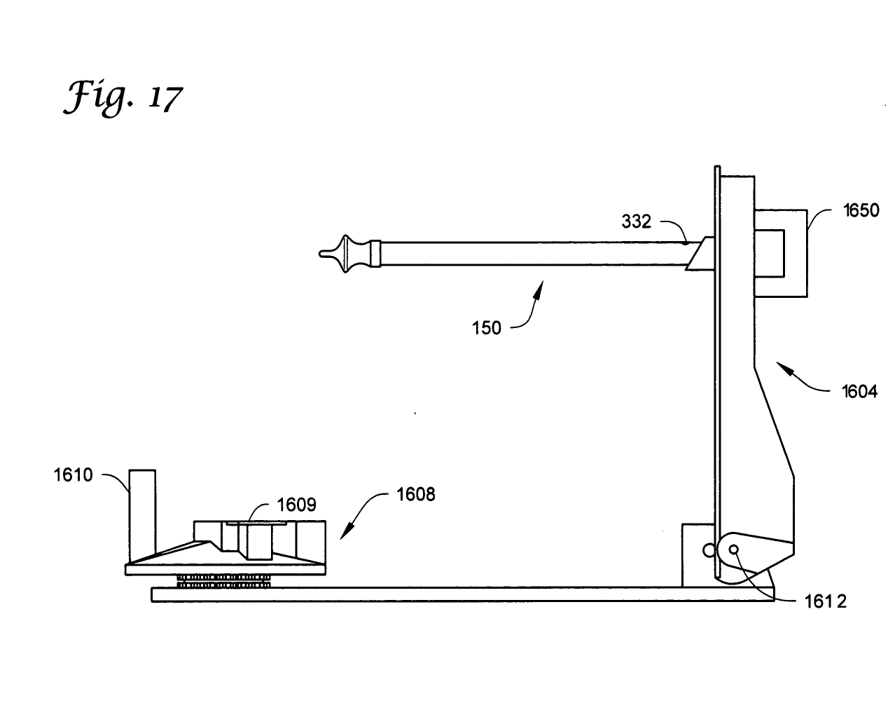

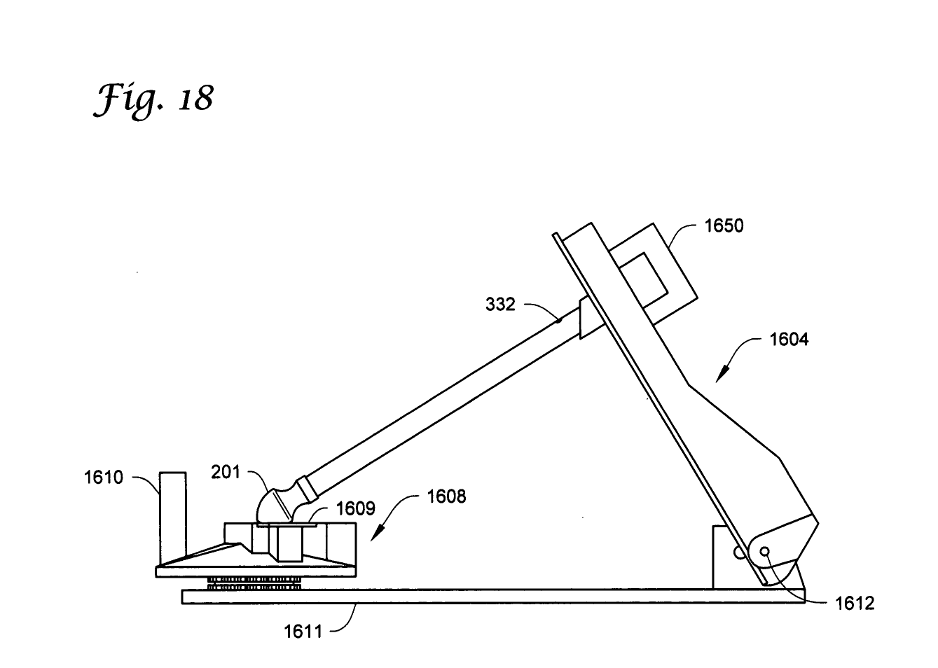

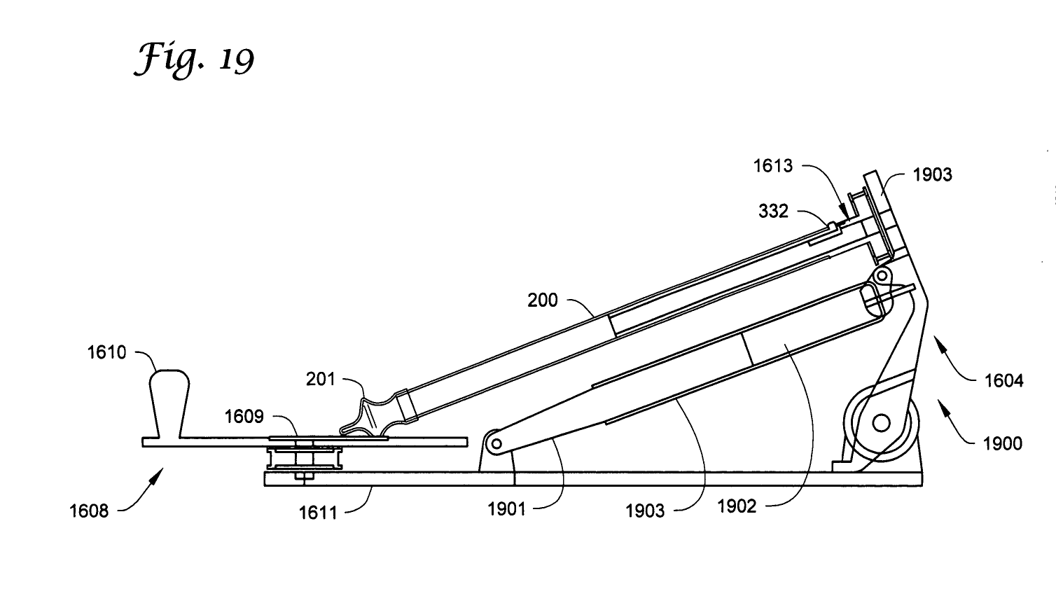

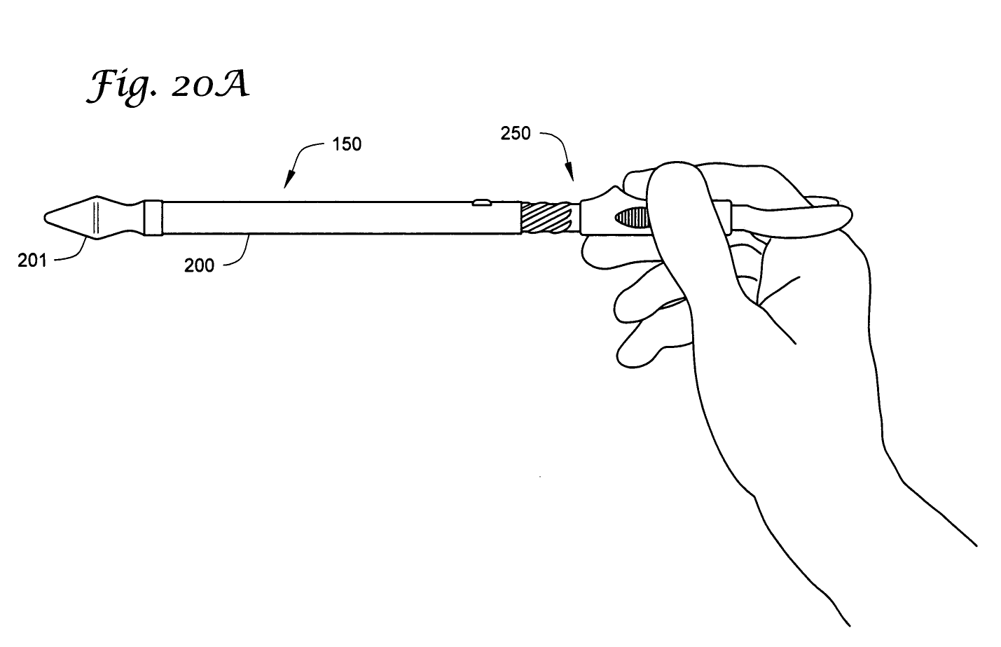

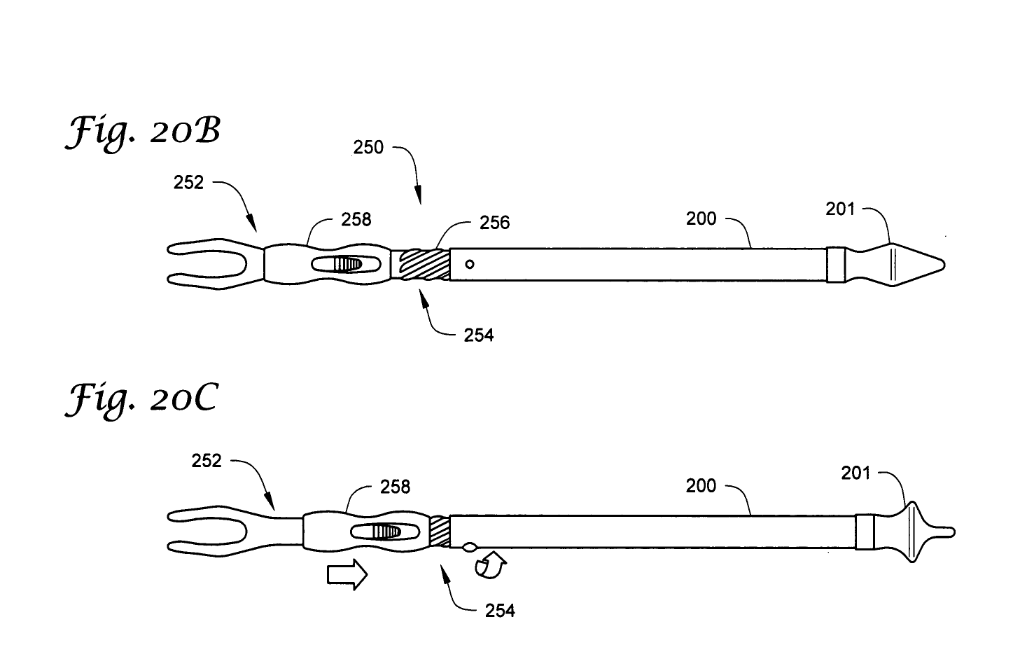

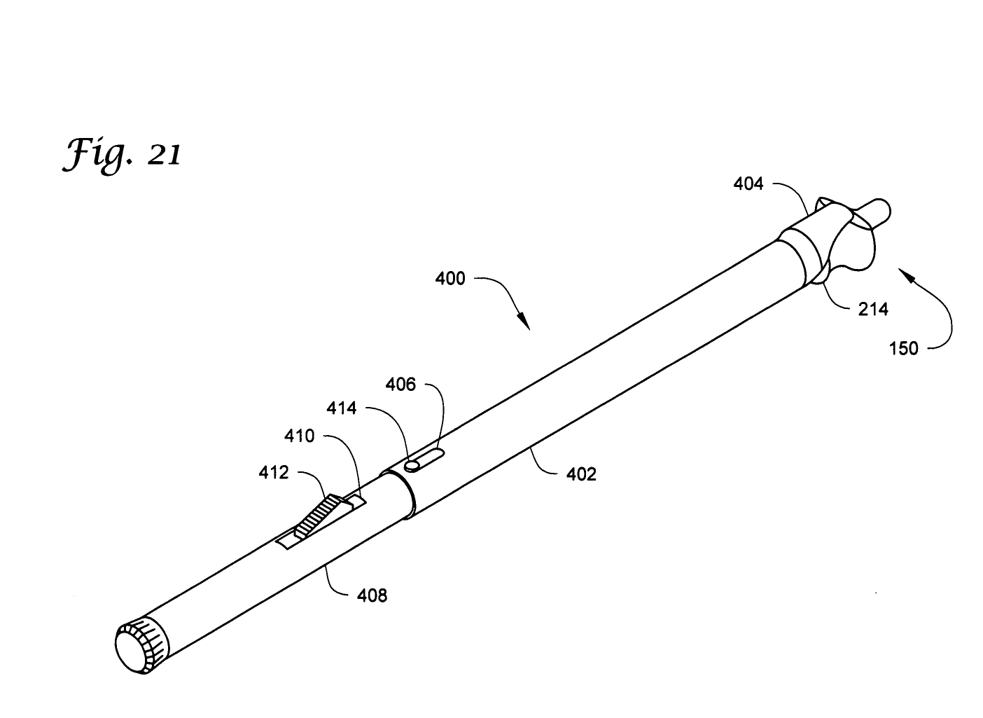

FIGS. 1 3A-C are detailed views of the tip region of the cell collector of FIG. 12 illustrating how expansion and rotation during collection occurs.

FIGS. 14A-C illustrates another example of a tip of a cell collector.

FIGS. 15A-B illustrate another mechanism for achieving cell cluster transfer.

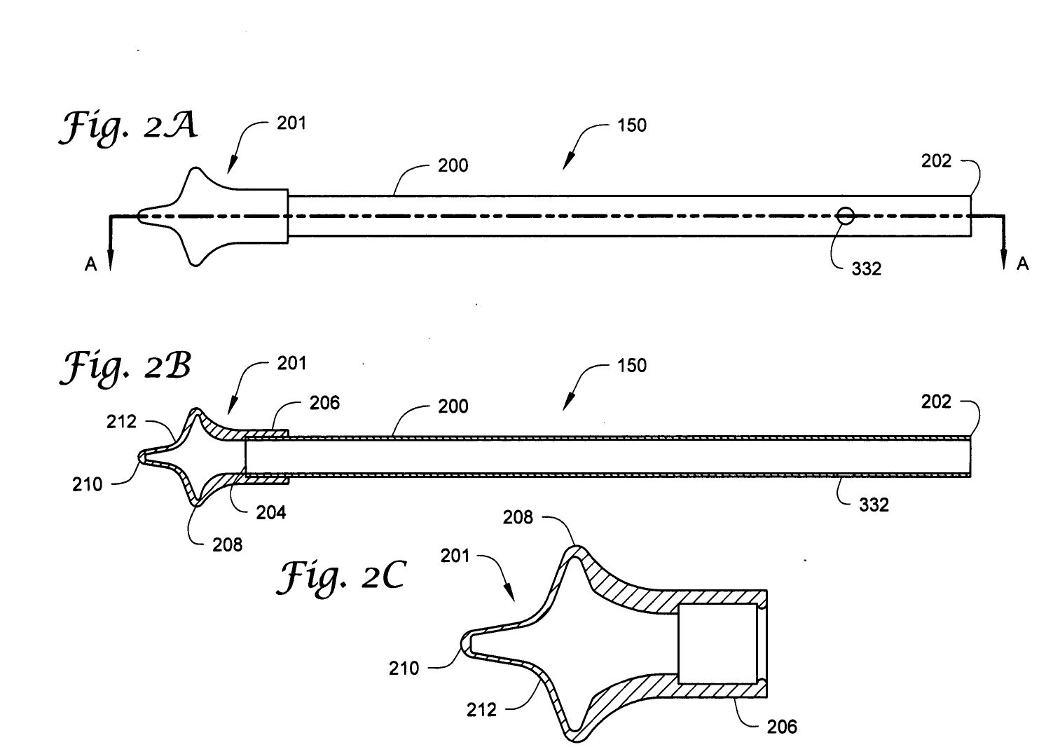

FIG. 16 illustrates how the cell collector of FIGS. 2A-B is attached to a mounting pulley of another embodiment of a mechanism for achieving cell transfer.

FIG. 17 illustrates the cell collector attached to the mounting pulley in FIG. 16.

FIG. 18 illustrates the cell collector and the mounting pulley during transfer of collected cell clusters onto a turntable having a slide.

FIG. 19 is a cross-sectional view of another embodiment of a mounting pulley for achieving transfer of cell clusters.

FIGS. 20A-C illustrate a mechanism for rotating the cell collector during collection.

### DETAILED DESCRIPTION

### I. Overview

Cancer is a disease of tissue not cells. Diagnosis of solid tumors by pathologists depends on recognition of the architecture of lesions, specifically how cells within a lesion differ from surrounding normal cells. The criteria used have included morphology, cytochemical stains to recognize cellular structures, and the use of antibodies and nucleic acid probes to determine the patterns of expression and organization of the cellular genetic material.

Neoplastic progression corresponds to accretion of genetic and epigenetic changes which render cells within the nascent tumor increasingly more able to proliferate without responding to normal regulatory signals and factors, invade surrounding tissue elements, become vascularized, and metastasize. However, at the earliest stage of this process, dysplastic lesions arise from the clonal expansion of a precursor cell which was minimally different from the surrounding normal cells.

Screening for cancer at its earliest stages requires recognition of rare dysplastic cells which have not clonally expanded to the point where the number of such cells is physically apparent or clinically manifested in symptoms. The means for recognition of such cells are limited with regard to morphological and biological criteria by the very nature of the minimal difference between these cells and the adjacent normal cells.

A concept of the invention is that a dysplastic precancerous lesion can be distinguished from normal tissue elements by analyzing clusters of cells to look for two or more biological markers that are rarely co-expressed in the same cell at any one point in normal tissue. For example, clusters of cells can be interrogated for two or more biological markers that indicate the existence of individual cells that are part of a local area where dysplasia may be present. The following description and examples refer to growth and apoptosis as the two or more biological markers for purposes of explaining the concepts. However, it is to be realized that two or more other biological markers that are rarely co-expressed in the same cell can be used, or could be used in conjunction with cell growth and apoptosis as markers.

The concepts described herein can be used to screen for dysplastic precancerous lesions from a number of regions of the body. For purposes of explanation, the inventive concepts will be discussed below with respect to the collection of clusters of cells from a cervix to screen for cervical cancer. However, it is to be realized that the inventive concepts can be used to screen for dysplasia by examining clusters of cells from other regions of the body, for example the bladder to screen for bladder cancer, the lungs to screen for lung cancer, the colon to screen for colon cancer, and the ovaries to screen for ovarian cancer. The clusters of cells can be collected from tissue, urine, induced sputum, cells washed from ovaries, and the like.

The clusters of cells are collected using a collector that is designed to enhance the ability of the collector to pick-up clusters or clumps of cells, and to facilitate transfer of the collected clusters of cells onto a receiving structure, for example a slide. In one embodiment, a combination of the material of the collector, the texture of the collection surface of the collector, and the use of expansion and rotation of the collector during collection facilitate the collection of cell clusters. Preferably, clusters of cells are transferred from the collector to the receiving structure in such a way as to retain the spatial relationships that existed between the cells in the clusters prior to sampling. Orientation marks on the collector and the receiving structure assist in maintaining the spatial relationship during transfer.

In the case of dysplastic lesions on the cervix, a cervical analysis system according to the concepts of the invention encompasses a cell collector, a receiving structure to which collected clusters of cells are transferred from the collector, reagents and a scanner device which together (1) obtain clusters of cells from the endocervical and ectocervical areas of the cervix; (2) maintain the spatial relationship among the collected cell clusters on the collector and when transferred to the receiving structure; (3) examine the molecular properties of the cell clusters to establish if there is any evidence of abnormality in the cells; and (4) do this in a manner that allows a clinician to ascertain where on the cervix a dysplastic lesion might be present.

The cervical analysis system is one embodiment of an approach to the screening of cell clusters present in specimens in order to identify dysplastic lesions by virtue of the application of biomarkers which reveal a characteristic imbalance in the biological properties of adjacent cells.

FIGS. 1A-C illustrate the concepts of cell cluster collection from a uterine cervix 50. FIG. 1A illustrates the cervix 50 formed by a uterus 52, with the cervix including a cervical canal 60, an endocervix 56, an ectocervix 62, and a transition zone 58 illustrated by shading that extends from the ectocervix to the endocervix. An exemplary lesion 54 is illustrated in the transition zone 58 at the endocervix 56 of the cervix.

FIG. 1B illustrates the concepts of a cell collector 100 that can be used to collect cells and cell clusters from the cervix 50. The collector 100 has a surface 104 that can conform to the contours of the cervix and which has properties such that clusters of cells from both the ecto- and endocervices 62, 56 are collected by the surface 104 to ensure collection of cell clusters from the transition zone 58, while preserving the spatial relationships among the collected cell clusters.

In addition, the collector 100 has a visible orientation mark 106 to permit the individual collecting the clusters of cells to orient the collector upon sampling of the cervix, and maintain that orientation upon subsequent transfer of cell clusters to a receiving structure 101 which also includes a corresponding orientation mark 108 as shown in FIG. 1C. Cell clusters can be transferred to the receiving structure 101 by contacting the surface 104 with the receiving structure 101 which is configured so that cell clusters transfer to the structure 101 rather than remain adhered on the surface 104. During transfer, the orientation marks 106, 108 are aligned, so that once transferred, cell clusters on the structure 101 have the same spatial relationship as they did on the collector 100. The cell clusters can then be analyzed to screen for potential abnormalities.

The cell collector 100 can have a number of different configurations as long as it is capable of collecting clusters of cells from both the endo- and ectocervices 56, 62 to ensure collection of cell clusters from the transition zone 58. In one embodiment, a combination of the material of the collector surface 104, the texture of the collector surface 104, and the use of expansion and rotation of the collector surface during collection facilitates the collection of the clusters of cells.

### II. Embodiment 1

A. Collection

With reference now to FIGS. 2A-C, details of a cervical cell collector assembly 150 embodying the concepts of the invention are illustrated. The collector assembly 150 includes a hollow tube 200 that is detachably connected to an expandable collection tip 201. The tube 200 is made from, for example, plastic or cardboard. The expandable tip 201, which is also the cell collection region of the collector 150, is a resiliently flexible structure that is made of an elastomeric material, for example a thermoplastic elastomer alloy such as Versaflex® CL30 available from GLS Corporation of McHenry, Ill. The expandable tip 201 preferably has a texture that enhances the ability of the collector to collect clusters of cells from the transition zone 58 upon expansion and rotation of the tip 201. For example, the tip 201 can have a texture of MT-11010. Other elastomeric materials could be used for the tip 201, for example microporous polyvinyl acetate, nitrile rubber, nitrile foam, urethane foam, silicone rubber, latex rubber, polyurethane and other elastomers having low durometer, high percent elongation and adequate texture to enhance collection of cell clusters.

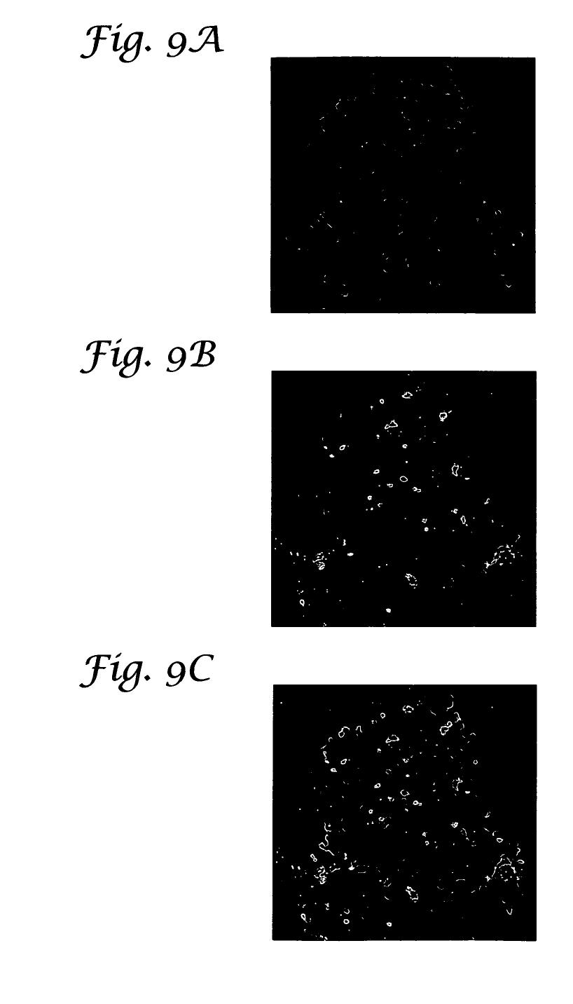

The tube 200 is generally hollow from one end 202 to the other end 204, with the end 202 of the tube 200 being open. With reference in particular to FIG. 2C, the expandable tip 201 in its as formed, original state includes a neck portion 206 detachably connected to the end 204 of the tube 200, a central enlarged shoulder 208, a tip region 210, and a transition section 212 extending between the shoulder 208 and the tip region 210. As shown in FIG. 9, an o-ring 214 can be provided around the neck portion 206 of the collection tip 201 to aid in retaining the tip 201 on the tube 200.

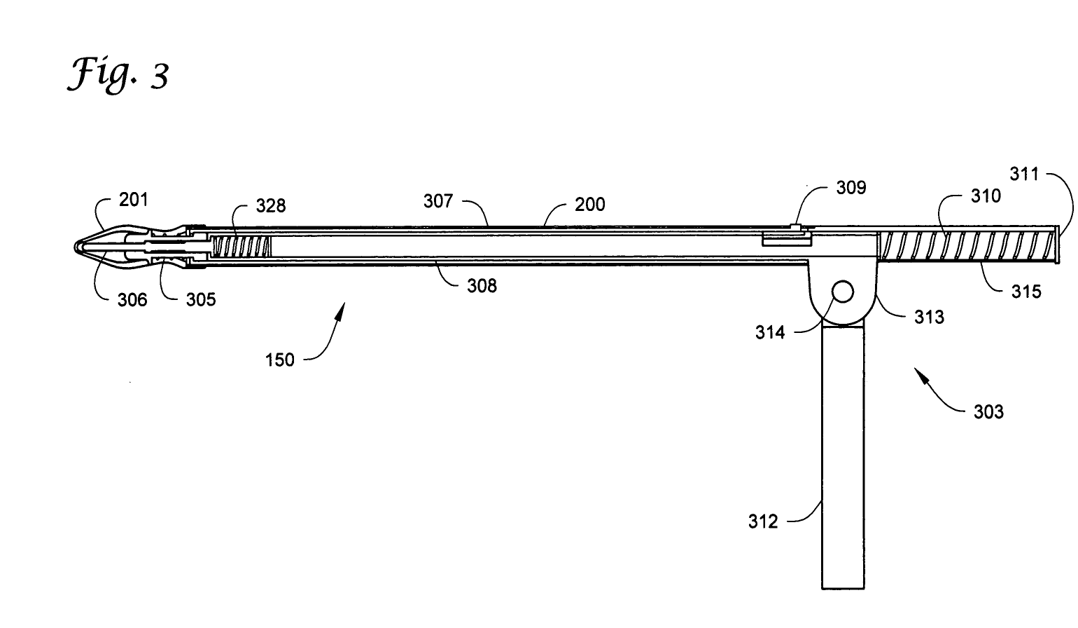

FIGS. 3-5 show the cervical cell collector assembly disposed on a collector handle assembly 303 for use in taking a cell sample. The assembly 303 includes an inner casing 308 and an outer casing 307, with the tube 200 being disposed around the outer casing 307, and the outer casing 307 being slidably disposed on the inner casing 308. A probe 306 projects forwardly from inside the inner casing 308 into the interior of the expandable tip 201. An expander probe 305 is disposed at the end of the assembly 303 surrounding the probe 306, with an end 320 of the probe 305 disposed in the outer casing 307 at the end of the outer casing 308. An opposite end 322 of the probe is enlarged and includes a shoulder 324.

The probe 306 can have a diameter of approximately 2 mm and project beyond the end of the expander probe 305 a distance between approximately 8 to 10 mm. The body of the expander probe 305 forward of the shoulder 324 can have a diameter of approximately 6 mm, while the shoulder 324 has a diameter of approximately 10 mm.

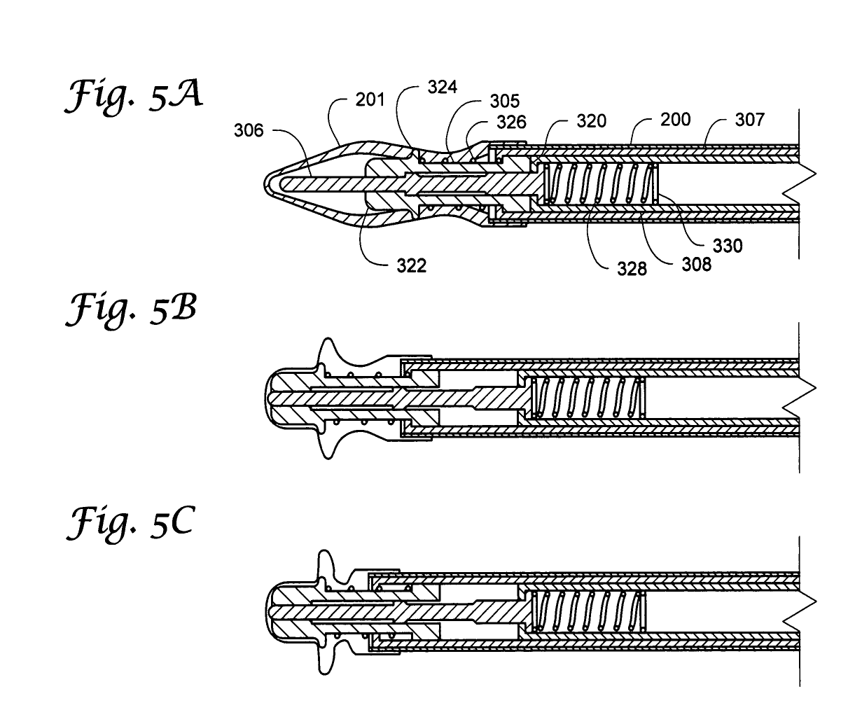

A coil spring 326 is disposed between the shoulder 324 and the end of the outer casing 307 for biasing the expander probe 305 to the left in FIGS. 3 and 5A-C. In addition, a coil spring 328 is disposed inside the inner casing 308 between the end of the probe 306 and a fixed ring 330 disposed in the inner casing. The spring 328 biases the probe 306 to the left in FIGS. 3 and 5A-C.

The outer tube 307 also includes a tube lock 309. The tube lock 309 comprises a resilient member fixed to the outer tube 307 that projects upwardly through an aperture 332 (see FIG. 2B) formed in the tube 200 of the collector 150. The tube lock 309 and aperture 332 cooperate to lock the tube 200 to the outer tube 307 of the handle assembly 303.

Returning to FIG. 3, a return spring 310 is disposed within the outer tube 307 between the end of the inner tube 308 and a spring cap 311 that is disposed at the end of the outer tube 307. The spring 310 biases the outer tube 307 toward the right in FIG. 3 while biasing the inner tube 308 toward the left, to return the outer 307 and inner tubes 308 to a home position shown in FIG. 3.

A handle 312 is fixed to a support 313 that is connected to the inner tube 308. The handle 312 is rotatably secured to the support 313 by a pivot 314 to allow the handle 312 to pivot between the position shown in FIG. 3 and a collapsed position where the handle 312 is generally parallel to the casings 307, 308. The outer tube 307 is formed with a slot 315 that allows relative sliding movements between the outer tube 307 and the support 313. The slot 315 extends to the right of the support 313 to the cap 311 in FIG. 3.

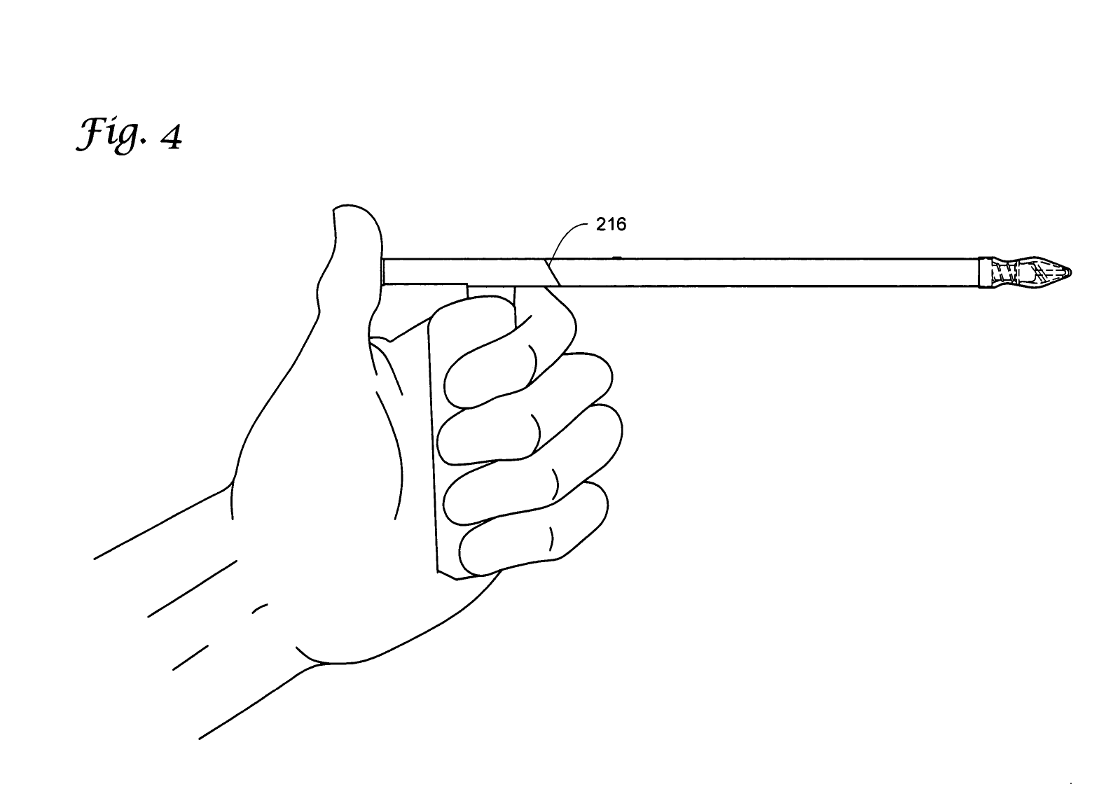

As best seen in FIGS. 3 and 4, the diameter of the outer tube 307 changes from a smaller diameter section that is designed to receive the tube 200 of the collector 150 to a larger diameter section adjacent the handle 213 and extending to the right of the support 313 in FIG. 3. The transition between the smaller diameter section and the larger diameter section forms a shoulder 216 (FIG. 4) against which the end of the tube 200 abuts. If desired, the end 202 of the tube 200 can be angled to match an angle formed by the shoulder 216 (see FIGS. 16-18). The angle of the shoulder 216 and the angle on the tube 200 can be aligned when the collector assembly 150 is slid onto the handle assembly 303 to help ensure that the collector assembly 150 is properly oriented on the handle assembly 303.

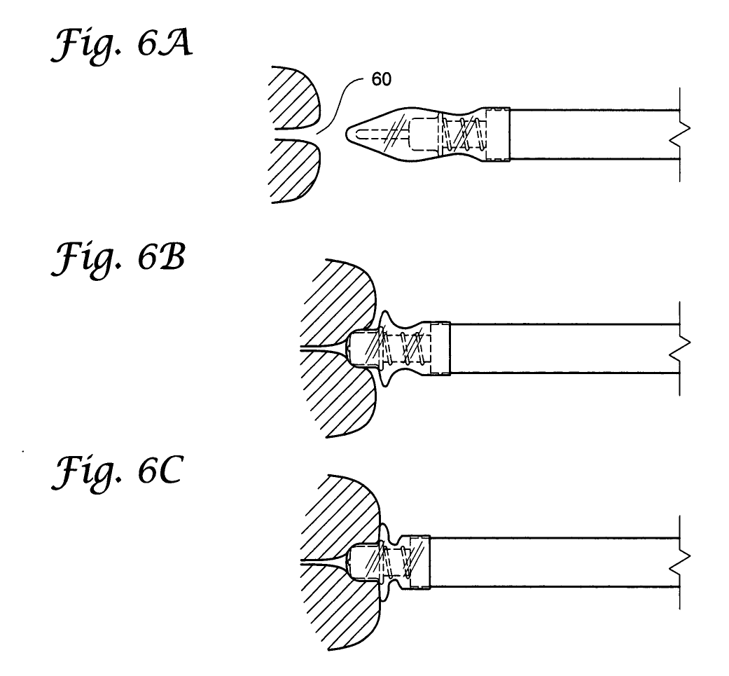

FIG. 4 is a schematic diagram of a hand holding onto the handle 312 with a thumb pressed against the spring cap 311. FIGS. 5A-C and FIGS. 6A-C, together with FIG. 4, show the process of collection using the cell collector assembly 150. The user initially inserts the cell collector assembly 150 onto the handle assembly 303. In doing so, the end of the probe 306 engages the tip region 210 of the expandable tip 201 causing the expandable tip to flatten out and temporarily reduce the shoulder 208 on the tip 201, as shown in FIGS. 5A and 6A. This improves the user's sight lines for inserting the collector into the cervix.

The user then pushes on the spring cap 311 with the thumb or other digit as shown in FIG. 4. This causes the outer casing 307 to be moved forward along with the expander probe 305, as shown in FIG. 5B. When the probe 305 moves forward, it causes the shoulder 208 of expandable tip 201 to expand outward from its flattened state, as shown in FIGS. 5B and 6B. The expander probe 305 bottoms out when it becomes flush with the end of the probe 306 after approximately 8 to 10 mm of travel, as shown in FIG. 5B. The expander probe 305 expands the endo-cervical canal to approximately 6 mm, with the expandable tip 201 in contact with the canal. Once the probe 305 bottoms out, continued pushing by the thumb continues movement of the outer casing 307 another approximately 3 to 4 mm, and at the same time pushes the tube 200 forward. As a result, the shoulder 208 and/or transition section 212 of the expandable tip 201 are compressed against the ectocervix 62 as shown in FIG. 6C.

During its movements, the expander probe 305 expands the tip region 210 of the expandable tip 201 into engagement with the endocervix 56. In addition, the shoulder 208 and/or transition section 212 of the expandable tip 201 compresses against the ecto-surface of the cervix 50. As a result, both endocervical and ectocervical cells, including cells from the transition zone 58, can be collected.

The expandable tip 201 is also rotated during collection in order to collect clusters of cells from the transition zone by shearing cell clusters from the transition zone 58 assisted by the texture of the tip 201. The tip 201 is rotated, for example, twenty to thirty degrees. The tip 201 can be rotated by the user manually rotating the handle assembly 303 and the collector assembly 150 connected thereto. Alternatively, the tip 201 can be rotated using a suitable mechanical rotation mechanism which causes rotation of the tip 201 once the tip region 210, shoulder 208 and transition section 212 of the tip 201 are expanded by the handle assembly 303 into contact with the endo- and ecto-cervices.

An example of a mechanical rotation mechanism is illustrated in FIGS. 20A-C. FIG. 20A illustrates the collector assembly 150 disposed on a handle assembly 250. The assembly 250 includes a U-shaped end portion 252, and an expansion and rotation portion 254 rotatably connected the U-shaped end portion 252 to permit rotation of the portion 254 relative to the end portion 252. The end of the portion 254 surrounded by the tip 201 is configured in a manner similar to that shown in FIGS. 5A-C. The opposite end of the portion 254 is provided with helical teeth 256 on the outer surface thereof.

A gripping sleeve 258 is slidably disposed on the portion 252 and the portion 254 over where the portions 252, 254 connect. Helical teeth (not shown) are disposed on the inside surface of the sleeve 258 for engagement with the teeth 256 on the portion 254.

During use of the assembly 250, after mounting the collector 150 onto the handle assembly 250, as the user inserts the probe, the probe 305 (shown in FIGS. 20A-C) is moved forward, causing the tip 201 to expand (FIG. 20B). Continued pushing by the user causes the tip 201 to expand further to engage against the ecto-cervix (FIG. 20C). The engagement with the ecto-cervix prevents further insertion, and causes the gripping sleeve 258 to move forward in the direction of the arrow in FIG. 20C. The sleeve 258 eventually moves far enough to contact the helical teeth 256. Continued advancement of the sleeve 258 and the engagement of the helical teeth causes the portion 254 together with the collector 150 to rotate as shown by the arrow in FIG. 20C.

After insertion, and expansion and rotation to achieve cell cluster collection, the pressure is released and the return spring brings the mechanism back to the original position. The tube lock 309 is depressed and the cervical cell collector 150 is then detached.

FIG. 21 shows another embodiment of a collector handle assembly 400 with the cell collector assembly 150 mounted thereon. The assembly 400 includes a front tube 402 having a deflector 404 connected thereto at the front end thereof. The handle assembly 400 is designed so that the tube 200 of the collector assembly 150 is slid into the tube 402 to mount the collector assembly 150. When the collector assembly 150 is mounted on the assembly 400, the deflector 404 flattens the shoulder 208 on the tip 201 to improve the sight lines for insertion during collection. The tube 402 also includes a slot 406 near the rear end thereof. The interior of the tube 402 around which the tube 200 is disposed is configured similarly as in FIGS. 5A-C.

The assembly 400 also includes a rear tube 408 having a front end thereof received within the rear end of the tube 402. A slot 410 is formed in the rear tube 408 and a button 412 is slideably disposed in the slot 410. The button 412 is connected to a projection 414 disposed within the slot 406 of the front tube 402.

The button 412 is illustrated in FIG. 20 at a home position, which is also the insertion position of the assembly 400. After properly inserted, the user pulls back on the button 412, and the button 412 moves to the end of the slot 410 to a rear button position. Since the button 412 is connected to the projection 414, the projection 414 also moves backward, which pulls the front tube 402 backward relative to the collector assembly 150 to release the deflection of the collection tip 201 caused by the deflector 404. Subsequently, the user pushes the button 412 forward to expand the collection tip 201. The button 412 is connected to the expansion mechanism shown in FIGS. 5A-C in such a manner that expansion occurs from the home position of the button to the forwardmost position of the button in the slot 410.

Once the button 412 is pushed all the way forwardly and the collection tip expanded, the tip is then rotated. The tip can be manually rotated, as discussed above, by manually rotating the rear tube 408. Alternatively, a suitable mechanical rotation mechanism can be provided for rotating the collection tip.

B. Transfer

After collection, the cell collector assembly 150 is mounted on a transfer device for use in transferring cell clusters from the tip 201 to a receiving structure for subsequent analysis of cell clusters. Examples of suitable receiving structures include a slide, a petri dish, and other structures to which cell clusters may be transferred for subsequent analysis of the cell clusters. The transfer device is constructed so that transfer occurs at equal pressures from receiving structure to receiving structure. Further, the surface of the receiving structure has greater adhesiveness than the surface of the tip 201 containing cell clusters to enhance the transfer of cell clusters from the tip to the receiving structure. When the receiving structure is a slide, the slide can be provided with a coating that results in the greater adhesiveness.

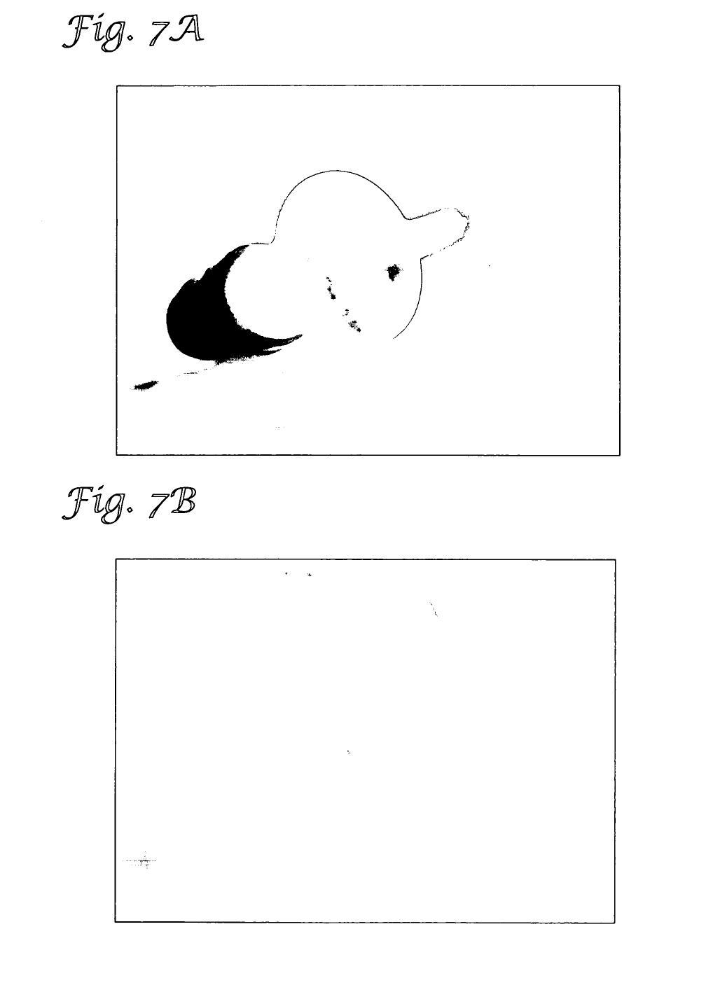

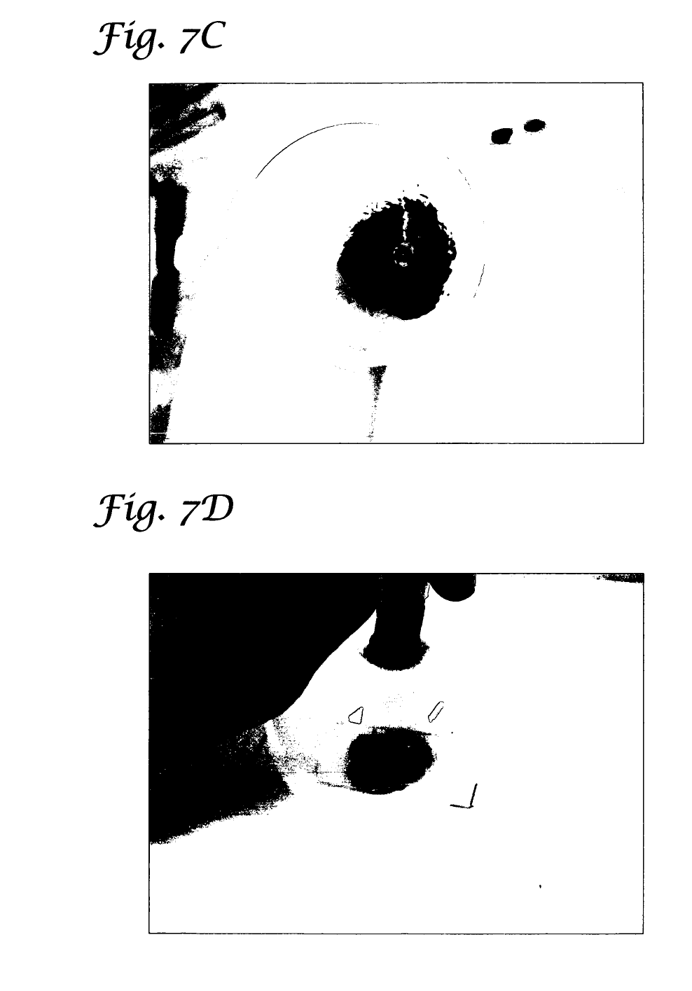

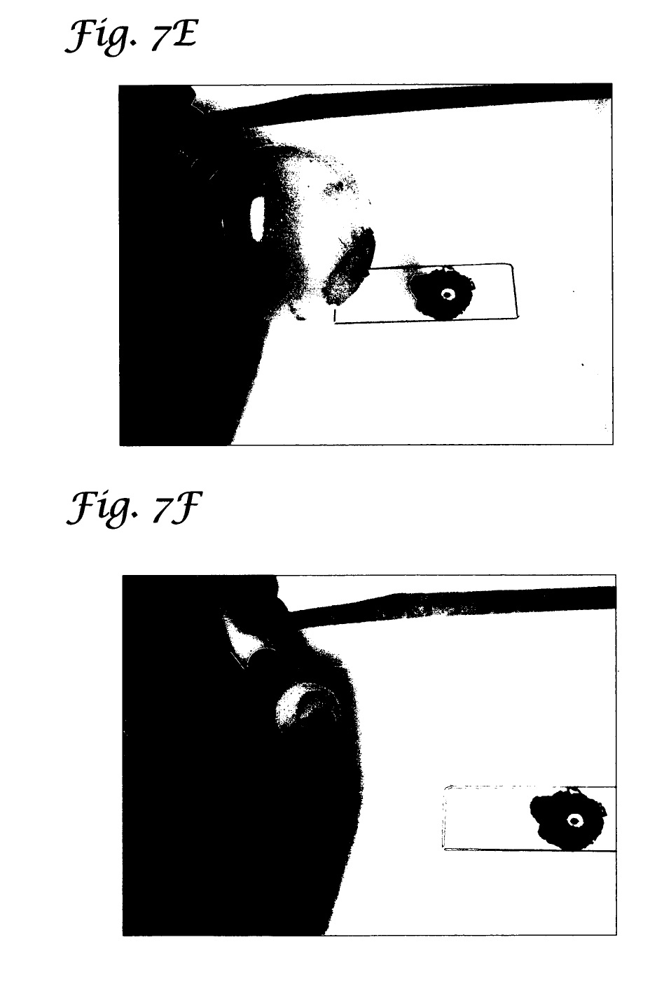

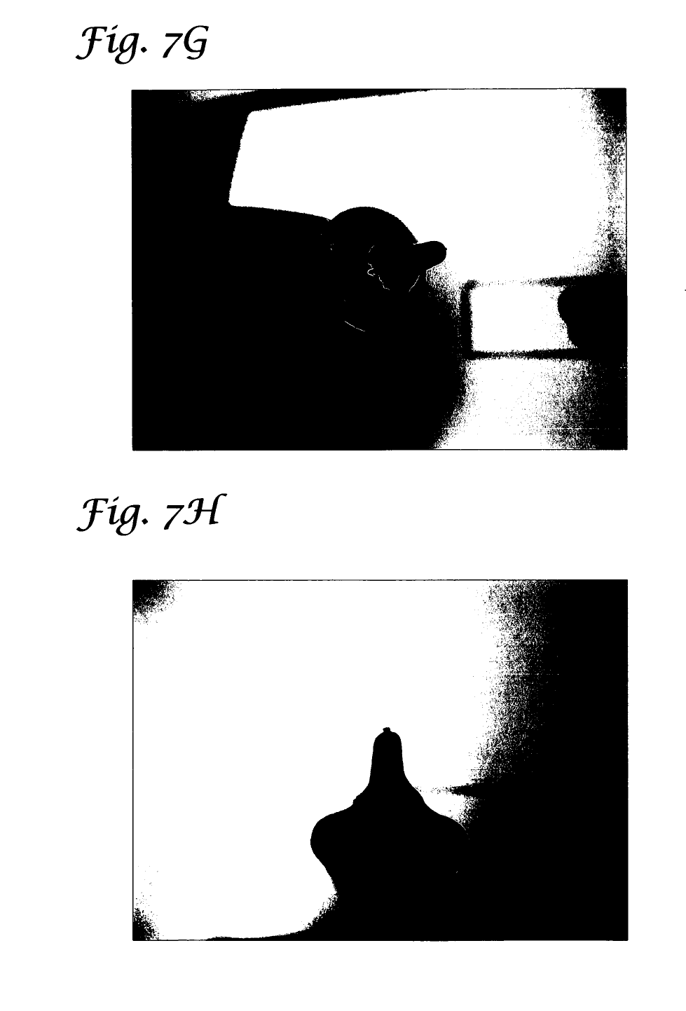

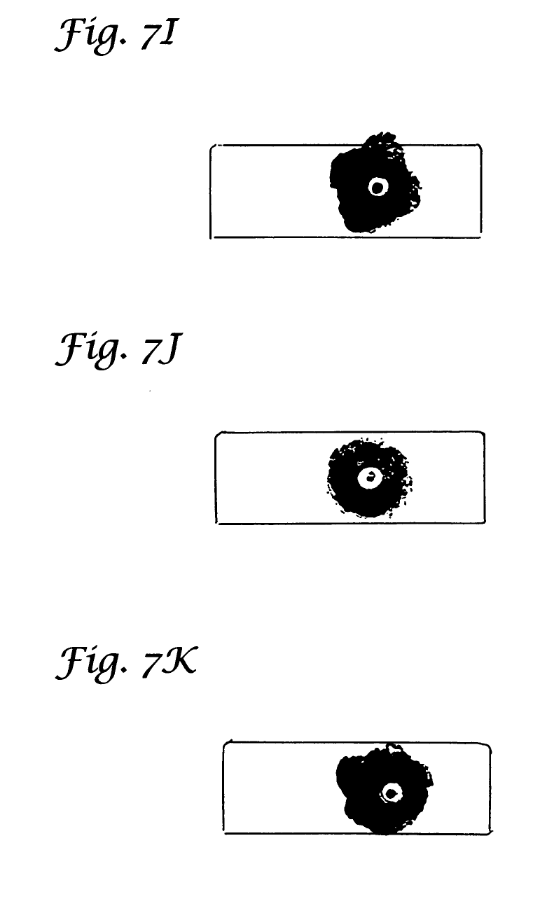

The tip 201 of the collector assembly 150 is preferably inflated using air during transfer. When the tip 201 is made from a thermoplastic elastomer alloy such as Versaflex® CL30, the elastomer allows uniform expansion of the tip during inflation. During inflation for transfer, the tip region 210 and the transition section 212 substantially go away (see FIG. 7B) so that the cell clusters on the tip region 210 and transition section 212 end up generally on a common plane for subsequent transfer of cell clusters to the receiving structure. This helps to maintain the spatial relationship of the cells in the cell clusters.

After transfer, the tip 201 can be removed from the tube 200 and put into a container with preservative to preserve remaining cell clusters on the tip 201. The tube 200 can then be discarded or connected to a new tip 201 for further collections. If the tip 201 does not need to be preserved, the tip 201 can be discarded.

FIGS. 7A-K illustrate the concepts of cell cluster transfer using the cell collector 150, with colored marker and marking fluid simulating collected clusters of cells. FIG. 7A shows a tip 201 of a collector with colored marker 500 on the tip indicating collected transition zone cell clusters. FIG. 7B shows the tip 201 inflated, with the colored marker 500 faint but still visible. FIG. 7C shows a marking fluid 502 added to the area that would contain the transition zone cell clusters to aid in visualizing transfer. FIG. 7D shows the inflated tip 201 being pressed down onto paper that is marked to represent the actual size of a slide 504. FIG. 7E shows the imprint that is left on the representative slide 504, with the imprint representing transferred cell clusters. FIG. 7F shows the tip 201 deflated to its original size and shape. FIG. 7G is a close-up view of the tip 201 showing areas where marking fluid (i.e. representing cell clusters) was and was not transferred. As shown in FIG. 7H, a small area of “cell clusters” did not transfer at the tip region 210 of the tip 201 and at the base of the transition zone 212. Critical transition zone “cell clusters” located between the tip region 210 and the transition zone 212 transferred fully. FIGS. 71-K illustrate the results of three separate transfers using marking fluid.

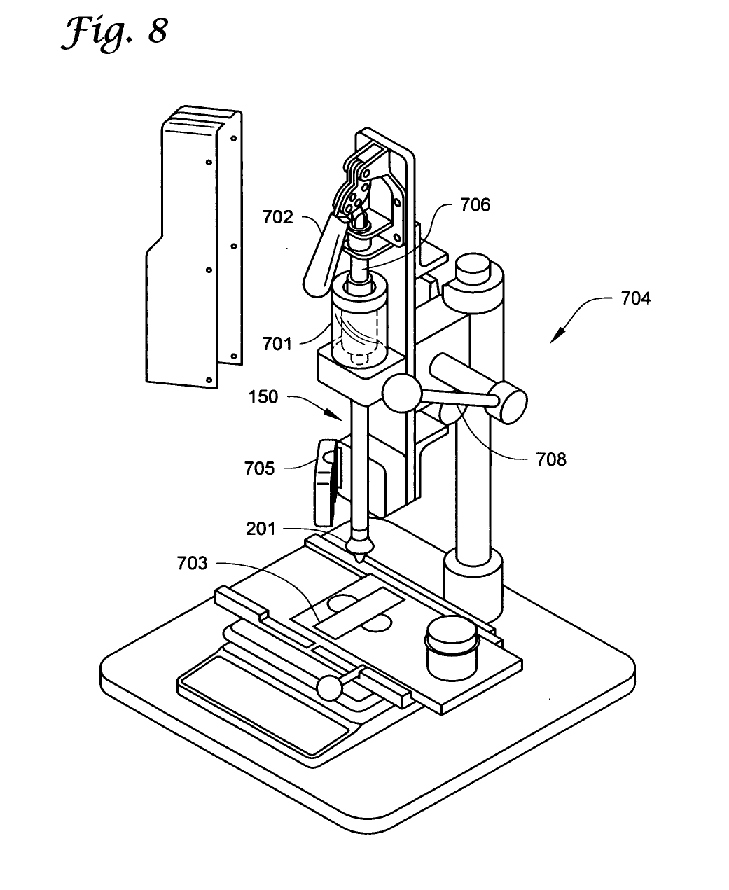

FIG. 8 shows an example of a cell cluster transfer device 704. In this example, the aperture 322 on the tube 200 of the cervical cell collector 150 acts as an orientation mark which is aligned with a corresponding mark on the transfer device 704 to orient the collector 150 on the transfer device. Correct orientation is necessary to maintain the relationship between any abnormal cervical cells recognized on the basis of their biological characteristics and the anatomic position of the suspicious areas from where the cell clusters were collected.

The collector 150 is placed on the device 704 such that the tip 201 faces a receiving structure in the form of a coated slide 703 placed on the bottom of the transfer device 704. The device 704 includes a clamp mechanism 705 that clamps the tube 200 and holds the tube 200 in place. The transfer device 704 also includes an air cylinder device 701 that is configured to pump air into the collector 150 in order to inflate the tip 201. A handle 702 is pivotally connected to the transfer device 704 and a rod 706 extends from the handle into the air cylinder device 701 for actuating a piston within the air cylinder device 701. As the user pushes down on the handle 702, the piston in the air cylinder device 701 is actuated to force air into the collector 150 through the tube 200 and into the tip 201 in order to inflate the tip (see FIG. 7B).

Once the tip 201 is inflated, a handle 708 connected to the device 704 is rotated. Rotation of the handle 708 causes the collector mount mechanism, including the collector assembly 150 mounted thereto, to move towards the slide 703 similar to a drill press. Eventually, the inflated tip is pressed down onto the slide 703, similar to the manner shown in FIG. 7D. The handle 708 is then rotated to retract the collector assembly 150, and the handle 702 released to deflate the tip 201.

FIGS. 16-19 show another embodiment of cell cluster transfer onto a receiving structure. A cell collector assembly is slidably disposed on a mounting arm 1613 of a mounting pulley 1604, as shown in FIG. 17. The mounting arm 1613 is hollow so as to allow air to pass through the rear end of the mounting arm 1613 and into the collector 150 for inflating the tip 201. The aperture 332 is aligned with a corresponding mark 1606 on the mounting arm 1613 to orient the cell collector on the arm 1613. The mark 1606 forms part of a lock for engaging with the aperture 332 to secure the collector onto the mounting arm 1613.

The mounting pulley 1604 includes a turntable 1608 having a handle 1610 and a support surface for receiving a microscope slide 1609. The slide 1609 is locked in place on the support surface using a suitable fixation mechanism, for example clamps. The turntable 1608 is rotatably mounted on a support plate 1611 to enable the turntable 1608 to rotate using the handle 1610. A support arm 1607 is pivotally connected to the plate 1611 by a pivot 1612, and the mounting arm 1613 extends from the support arm 1607. In addition, an air pump 1650 is connected to the support arm 1607 and is fluidly connected to the rear end of the mounting arm 1613 for pumping air into the mounting arm 1613 for inflating the tip 201. The air pump 1650 could be motor driven or driven manually by the user.

After collection, the collector is mounted on the mounting arm 1613 and locked in place (FIG. 16). The support arm 1607 is then rotated downwards counterclockwise toward the slide 1609 on the turntable 1608 (FIG. 17). Prior to contacting the slide, the collector tip 1601 is expanded to an appropriate volume by the air pump 1650. As shown in FIG. 18, the tip 201 is oriented correctly on the slide 1609 at the proper angle for cell cluster transfer.

FIG. 19 illustrates an alternate implementation of an air pump, where the tip 201 is expanded using a plunger 1901 and a plunger chamber 1902 defined by a plunger body 1903. The plunger chamber 1902 is in fluid communication with the back of the mounting arm 1613 such that when the support arm 1607 is rotated counterclockwise toward the turntable 1608, the plunger 1901 and plunger body 1902 compress, forcing air out of the plunger chamber 1902 and into the back of the mounting arm 1613 to inflate the tip 201 as the tip is rotated down toward the slide 1609.

Once the tip 201 is rotated down into engagement with the slide 1609, the support arm 1607 is locked to retain the tip 201 in contact with the slide 1609. The turntable 1608 is then rotated using the handle 1610. A drive mechanism is connected between the turntable 1608 and the mounting arm 1613, which is rotatably mounted on the support arm 1607, to cause rotation of the mounting arm 1613 and the collector 150 fixed thereto. The drive mechanism is configured such that once the collector tip 201 makes one full revolution, a spring in the turntable 1608 returns the mechanism back to the original position.

As shown in FIG. 1C, FIG. 8 and FIGS. 16-19, a slide is utilized as a receiving structure to which cell clusters are transferred. The slide is preferably constructed such that (i) part of or its entire surface is treated with a coating so that cells and cell clusters will adhere to the slide rather than remained adhered to the surface of the collector; and (ii) it can be uniquely oriented with respect to the orientation mark on the collector. These characteristics can be achieved by a surface modified glass that is painted or etched so that patients may be identified and the corresponding collector registered.

C. Analysis

The cells and cell clusters transferred onto slide 101 as shown in FIG. 1 are fixed. The fixative can be any fluid or aerosol that will preserve the shape and biochemical characteristics of cervical cells. The fixative can be one of the fluids or aerosols currently used to fix cytologic specimens, or modifications of these formulations which enhance the preservation of cell structure or the ability to process the material for other applications, such as reaction with molecular probes. One such example of an aerosol fixative is Shandon CytoFix.

In order to label the cells and cell clusters, staining reagents including one or more molecular probes that react with a biomarker characteristic of dysplastic cervical epithelium could be used. The biomarkers that can be assessed include proteins, especially modified or activated forms of molecules expressed by proliferating cells. FIGS. 9A-C illustrate one example, where cervical cells, treated with a M344, an inhibitor of histone deacetylase which causes imbalanced cell cycles in neoplastically transformed cells, have been stained with a protein expressed in proliferating cells, phosphorylated ribosomal protein S6 (FIG. 9A) and cleaved cytokeratin 18 (FIG. 9B) which is a specific marker of apoptosis. This is one pair of markers expressed in dysplasia of the cervix, but not in the same cells within a lesion, as illustrated in the merged image (FIG. 9C). Other pairs of markers could be used, including markers of proliferation and cell cycle inhibitors. Examples of proliferation markers are Ki-67 antigen and proliferating cell nuclear antigen (PCNA). Cell cycle inhibitors, which are not normally expressed at high levels in actively growing cells, include p16, p21, and p27. The successful application of any given pair of markers using the screening methods described herein will depend upon the particular biological features of the tissue and neoplasm to which it is to be applied.

Other biomarkers that can be used include nucleic acids, messenger RNA molecules for genes whose expression is enhanced in dysplastic cervical cells, lipids and glycosylated forms of proteins and lipids. The functions of these target biomolecules in proliferating and dysplastic cells can include intracellular signal transduction receptors (e.g., mitogen-activated protein kinases), structural proteins (e.g., cytokeratins), and nuclear proliferation-related gene products (e.g. Ki-67). The expression of these proteins can be a function of, for example, aberrant growth or apoptosis.

The manner in which the staining reagents are applied and detected in order to ascertain the expression of such biomolecules can include modification of antibody and nucleic acid probes with fluorophores (e.g. FITC), reactive tags (e.g. biotin), or direct conjugation of the molecule with a reporter molecule (e.g. horse radish peroxidase). Detection of these probes can be directly (e.g. by epifluorescent illumination) through the reaction with an enzymatic reporter molecule (e.g. streptavidin-conjugated alkaline phosphatase) and/or addition of precipitating substrates (e.g. nitro blue tetrazolium and bromochloroindolyl phosphate) for a calorimetric readout.

In order to recognize the presence of groups of dysplastic cells indicative of a cervical intraepithelial lesion, some manner of counterstain can be employed. This can be achieved using reagents currently employed in immunocytochemistry and immunohistochemistry to facilitate the visualization of cells (e.g. methyl green or hematoxylin), reagents reacting with a major cellular feature (e.g. phalloidin), or the reagents used to develop what is commonly termed a Pap stain.

A scanning device is subsequently used to measure the intensity of the individual signals from the appropriately detected probes and determine how the ratio of these signals varies across the collected and stained sample. The scanning and analysis are integrated over an area approximating the smallest preneoplastic lesion that is morphologically apparent to a clinician and which can be confirmed by histology or immunohistochemistry. The scanning device may be automated to permit multiple slides to be analyzed and the necessary analytic software can be either resident in the scanner or present on an external computer.

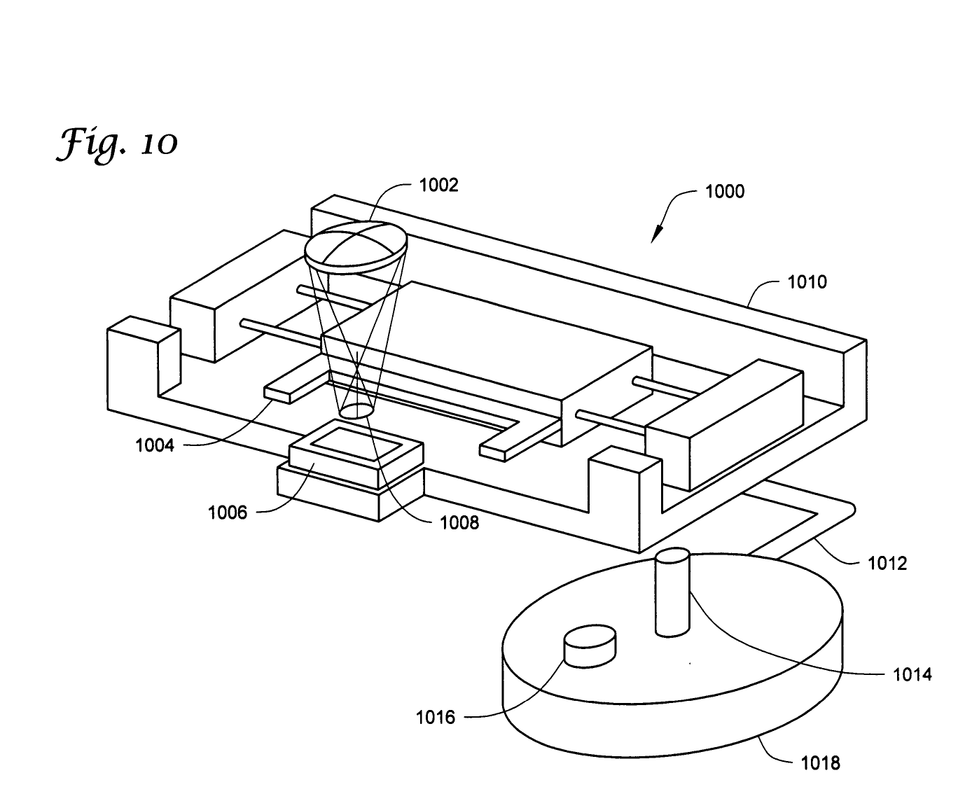

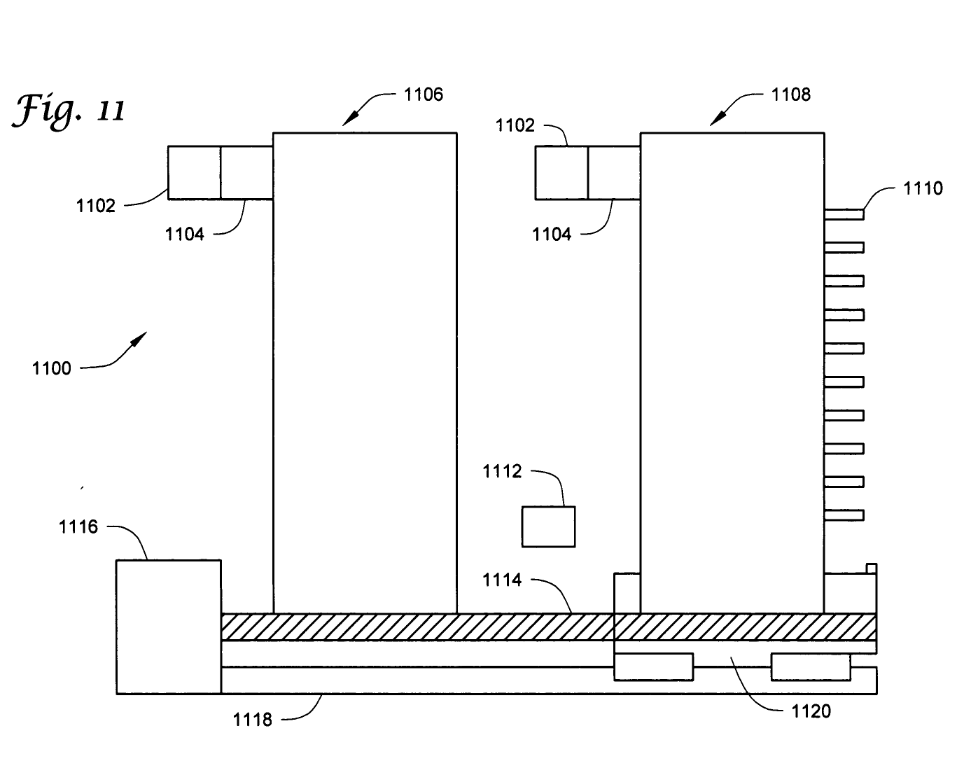

FIG. 10 depicts one example of a scanner device 1000 that can be used for reading the specimen slides. The scanner device 1000 shown in FIG. 10 is a manual instrument. The manual instrument in this example comprises a conventional magnifier 1002 (e.g. 3× or higher) mated to a solid state planar illuminator 1006. An exemplary value for the field of view of the magnifier is 8 mm, although other values can be used depending on the needs of the user.

A cantilevered two-axis manual stage 1010, with a slide holder 1004 connected thereto, allows for the positioning of a slide between the magnifier and the illuminator. A differential 4-bar linkage 1012 is provided to allow for both coarse and fine slide positioning under the magnifier. The linkage 1012 is connected to a stage positioner 1018 that includes a joystick 1014 and lock 1016. A slider 1008 containing excitation and emission filters is provided to allow the specimen to be viewed in both white light and fluorescence. The slider 1008 is inserted between the planar illuminator and the slide holder. The manual device 1000 could also have a focusing nob to allow the user to adjust the resolution of the magnifier 1002.

A battery or wall wart can be used to power the illuminator. The illumination provided from the illumination unit 1006 will depend on the excitation light intensity needed to saturate the fluorophore used and the emission intensity produced by a positive cell cluster. Exemplary values include a dye with an absorption (excitation) maximum of 495 nm and emission maximum of 519 nm, or absorption at 590 nm with emission at 617 nm.

FIG. 11 depicts another example of an instrument 1100 that can be used for reading the specimen slides. The instrument 1100 shown in FIG. 11 is an automated unit. The automated unit employs a “contact image sensor” (CIS) 1112 for capturing slide images and a vacuum chuck mounted on a ball slide to shuttle slides 1110 past the CIS 1112 enroute between an input elevator 1108 and an output elevator 1106. The elevators 1106, 1108 are driven by motors 1102 and gears 1104.

A taut-band drive and a lead screw drive 1114 driven by a motor 1116 are two examples of devices that would could be employed as a shuttle 1120. The shuttle 1120 rides on a linear bearing 1118. The elevators 1106, 1108 are one example of a moving belt design. In another example, the elevators could have a vertical walking beam design. The choice of elevator is dependent on packaging constraints and on throughput/batch size requirements. The scan time will depend upon light levels and the specific CIS used.

The CIS will also be responsible for reading barcode data from each slide. The barcode will include patient demographic data that can be printed in reports. Providing barcode data will decrease errors due to manual handling. Also, positive sample ID is mandatory for CLIA compliance.

A typical CIS reader has the ability to capture the barcode and decoding software (e.g. 8-10 characters of Code 128). A 200 DPI CIS (e.g. P1216MC-DR from Peripheral Imaging Corporation) can be used in the system. If there is a requirement for a specific wave length and gray scale, then it is contemplated that other CIS modules can be used. 200 DPI and higher monochrome and color CIS modules are available from numerous suppliers. If necessary, the CIS module can be modified for the particular application. For instance, it might be desirable to add wavelength selection filters. It also may also be desirable to remove the cover glass or go with a fractional pitch GRIN lens bar. It also may be desirable to use two monochrome CIS modules, one for each color, rather than cleaning up the spectral responses in a single color module. Again, the illumination provided by the CIS will depend on the excitation light intensity needed to saturate the fluorophore used and the emission intensity produced by a positive cell cluster. Exemplary values include a dye with an absorption (excitation) maximum of 495 nm and emission maximum of 519 nm, or excitation at 590 nm with emission at 617 nm.

The automated unit can be controlled in one example with a single board computer (SBC) that is specifically designed for use in embedded systems rather than in desktop/laptop applications. Exemplary SBC's include but are not limited to those produced by Sharp, Atmel and Auron. The SBC will also be responsible for data acquisition/processing and printing. The SBC will have to be programmed, in a known manner, for the specific application of controlling the automated system and acquiring and processing the data received from the CIS. If significant user interface interaction is required, such as showing the results of all samples in a window, complex printing, or storage of raw data, the data could be transferred to a personal or mainframe computer by using a USB interface, or similar mechanism of data transfer.

The power source used for the automated system can take many forms. In one example, rechargeable batteries could be used. The power requirement of a processor and LCD display at 5 VDC is ˜450 mA. Such a power requirement could be met with NiMH type batteries. For instance, four 3500 mAHr batteries would provide 7 hrs of operation on a new battery. If AA batteries, in the 1500 mAHr range were used, then 3 hours of operation would be provided on a new battery.

Finally, it is contemplated that a “lite” version of the automated system could also be effected. Such a lite version would include a LCD screen, a one axis stage, a CIS sensor, and scaled down processing capability. The user would position the slide under the CIS. The user could then push a button to acquire the data and the data would then be displayed on the LCD screen.

### III. Embodiment 2

With reference now to FIGS. 12 and 13, another embodiment of a cervical cell collector 10 for collecting cells in a uterine cervical canal 100 is illustrated. In this example, the cervical cell collector is comprised of an assembly that includes a flexible cell sampling region 12 and abutting rigid pusher 22 within which is contained a second assembly consisting of a tip expander 16 rotatably mounted on a rigid core element 14 with one set of features 31 of the tip expander engaging corresponding actuating features 32 of the core element 14 and a second set of features 33 engaging mating features of the pusher 34. The actuating features 32 of the core element 14 are configured, by way of example, as a screw thread having a suitable pitch. A stylette 18 attached to the core element 14 passes through an opening 20 in the tip expander 16.

The cell sampling region 12 can be a resiliently flexible structure that is made of a suitable elastomeric material such as microporous polyvinyl acetate, thermoplastic elastomer, nitrile rubber, nitrile foam, urethane foam, silicone rubber, latex rubber, polyurethane or any material having suitable low durometer, high percent elongation and surface qualities.

As suggested by FIGS. 13A, 13B, and 13C, the cervical cell collector can transition between an extended state (FIG. 13A); an intermediate state (FIG. 13B); and a collapsed state (FIG. 13C). The clinician guides the tip of the cervical cell collector 10 in its extended state into the cervical canal 100 to the desired depth (indicated as the tip depth) as shown in FIG. 13A. In this state, the pusher 22 is retracted and the cell sampling member 12 is approximately conformal to the exterior surface of the tip expander 16. Once the clinician has properly positioned the tip of the cervical cell collector 10 in the cervical canal 100, the pusher is advanced toward the cervical os while the core element 14 and stylette 18 remain stationary. As features 31 and 34 of the pusher 22 are engaged with corresponding features 32 and 33 of core element 14 and tip expander 16, respectively, advancing the pusher 22 causes the tip expander 15 to likewise move toward the os and to rotate around stationary core element 14. Concurrently, advancement of pusher 22 applies a compressive force to the cell sampling member 12 thereby causing it to deform radially outward against the exterior portion of the cervical os as is shown in FIGS. 13B and 13C. Advancement of the tip expander 16 into the tip of the cell sampling member 12 causes the diameter of tip of the cell sampling member 12 to increase, thereby pressing the exterior surface of the cell sampling member 12 against the walls of the cervical canal 100. The rotary motion of the tip expander 16 relative to the interior surface of the cell sampling member 12 facilitates entry of the tip expander into and, thereby, the expansion of the cell sampling member.

Contact and rotation of the cell sampling member 12 against the surfaces of the cervical os and canal 100 causes exfoliated cervical cells to adhere to the exterior surface of the cell sampling member. Retraction of the pusher 232 withdraws the tip expander 16 from the tip of the cell sampling member 12, thus allowing the cell sampling member to return to its initial extended state. The cervical cell collector 10 may then be removed from the cervical canal 100 and vagina and the cells collected on the surface of the cell sampling member prepared for evaluation.

The cells captured on the cell sampling region 12 may be prepared for evaluation by several means. One such means is the preparation of a suspension of the capture cells in a suitable preservative medium by immersing and, preferably, agitating the cell collection surface in the preservative medium. The cells of the resulting suspension may be deposited upon a microscope slide or similar surface in the manner of a conventional monolayer cell preparation and stained and evaluated in accordance with established methods. Alternatively, the suspended cells may be evaluated using a flow cytometer.

Another means of preparing the captured cells for evaluation is schematically illustrated in FIGS. 14 and 15. In this means, a rigid mandrel 114 is inserted into the cell sampling region 12 to force those portions of the cell sampling region to which cells are adhered to assume the shape of the mandrel as shown in FIG. 14. Mating keying features 112 and 116 on the cell sampling region 12 and mandrel 114 ensure that the cell sampling region maintains a defined orientation with respect to the mandrel. This may be accomplished in a manner such that the imprint on the slide corresponds to a particular mark on the collector, in order to reflect the orientation of the device as it had been inserted into the cervical canal. Such imprinting permits the clinician to accurately identify the region from which the cells originated.

As shown in FIG. 15, the cell-bearing surface of the cell sampling region may then be brought into contact with a microscope slide or similar, and appropriately treated, surface and rolled across this surface along a suitable arc such that the entire cell-bearing surface of the cell sampling region is brought into contact with a microscope slide. Contact of the cell-bearing surface of the cell sampling region with the microscope slide causes cells to be transferred from the cell sampling surface to the microscope slide. These transferred cells may then be stained and evaluated in accordance with established methods. In this method, the relative spatial locations of the cells are preserved, thus allowing the approximate location on the cervix from which the cells were collected to be determined. The slide is generally coverslipped. The composition of the mounting medium employed will be determined by whether or not the material on the slide will be treated in some other way after examination of the cervical analysis system result.

The coverslipped slide is reviewed using the appropriate illumination. It is scrutinized to determine whether there is a localization of signal in one area of the cervical sample. The location of the candidate lesion is noted with respect to a map of the cervix indicating the locations of the collected cells.

Cells of the ecto- and endocervices are sampled using a collector with the characteristics described above. The sample may be collected by a physician or health care worker. Alternately, it should be possible to train women to collect their own samples to achieve the purposes of the cervical analysis system.

While the invention has been described in conjunction with a preferred embodiment, it will be obvious to one skilled in the art that other objects and refinements of the present invention may be made with the present invention within the purview and scope of the present invention.

The invention, in its various aspects and disclosed forms, is well adapted to the attainment of the stated objects and advantages of others. The disclosed details are not to be taken as limitations on the invention.

What is claimed is:

1. A system for detecting abnormal tissue in a cervix comprising:
a collector for collecting spatially arranged clusters of cells from an ectocervical region and an endocervical region of a cervix, the collector maintaining the spatial integrity of the clusters of cells collected on the collector;
a receiving structure for receiving clusters of cells transferred from the collector, wherein the receiving structure and the collector are configured to maintain the spatial integrity of the clusters of cells transferred to the receiving structure;
an assay for preparing the clusters of cells transferred to the receiving structure for examination; and
a scanner device for detecting the clusters of cells prepared by the assay, so as to detect whether there may be an imbalance of two or more cell expression properties exhibited in a state of dysplasia.

2. The system of claim 1, wherein the collector and the receiving structure each include at least one orientation indicator disposed thereon so as to assist with maintaining the spatial integrity of the collected and transferred cells respectively.

3. The system of claim 1, wherein the collector further comprising a main body; and a resilient surface disposed proximate one end of the main body, the resilient surface having a contact texture being suitable for collecting clusters of cells from the ectocervical and endocervical region of a cervix, the resilient surface being made of a material that allows uniform expansion of the resilient surface, and the resilient surface may be rotatable,
wherein when the resilient surface is expanded and rotated, the contact texture of the resilient surface enhances the collection of clusters of cells from the ectocervical and endocervical regions by the resilient surface.

4. The system of claim 3, wherein the material of the resilient surface comprises a thermoplastic elastomer alloy.

5. The system of claim 3, wherein the contact texture comprises MT-11010.

6. The system of claim 1, wherein the assay further comprising a cytological detection cocktail that includes a first reagent as a marker for first cell expression property and a second reagent as a marker for a second cell expression property.

7. A system for detecting abnormal tissue comprising:
a collector for collecting clusters of cells from a local area of tissue where a potential imbalance of two or more cell expression properties may be exhibited in a state of dysplasia;
a receiving structure for receiving clusters of cells transferred from the collector;
an assay for preparing the clusters of cells transferred to the receiving structure for examination; and
a scanner device for detecting the clusters of cells prepared by the assay, so as to detect whether there may be an imbalance of two or more cell expression properties exhibited in a state of dysplasia.

8. A method for detecting abnormal tissue in a cervix, the method comprising:
(a) collecting clusters of cells from an ectocervical region and an endocervical region of the cervix using a collector, while maintaining the spatial integrity of the clusters of cells collected on the collector;
(b) transferring at least some of the clusters of cells collected by the collector to a receiving structure, while maintaining the spatial integrity of the clusters of cells transferred to the receiving structure;
(c) applying a cytological detection cocktail to the clusters of cells transferred to the receiving structure; and
(d) analyzing the clusters of cells for an imbalance of two or more cell expression properties.

9. The method of claim 8, wherein the spatial integrity of the collected and transferred clusters of cells being maintained by at least one orientation indicator on the collector and the receiving structure, respectively.

10. The method of claim 8, wherein the collecting step further comprising contacting clusters of cells at the ectocervical and endocervical regions, the clusters of cells being contacted with a resilient surface of a collector, the resilient surface having a contact texture being suitable for collecting clusters of cells from the ectocervical and endocervical regions;
expanding the resilient surface of the collector; and
rotating the resilient surface with respect to the ectocervical and endocervical regions;
wherein when the resilient surface is expanded and rotated, the contact texture of the resilient surface enhances the collection of clusters of cells from the ectocervical and endocervical regions by the resilient surface.

11. The method of claim 10, wherein the expanding step includes mechanically expanding the collector.

12. The method of claim 10, comprising manually rotating the resilient surface.

13. The method of claim 10, comprising mechanically rotating the resilient surface.

14. The method of claim 10, comprising rotating the resilient surface approximately 20 to 30 degrees.

15. The method of claim 10, comprising rotating the resilient surface after expanding the resilient surface.

16. The method of claim 8, wherein the transferring step further comprises pneumatically expanding the collector.

17. The method of claim 16, wherein the collector is expanded to an extent such that cell clusters from the ectocervical and endocervical regions are generally on a common plane.

18. The method of claim 8, wherein the step of applying the cytological cocktail further comprising applying a first reagent as a marker for first cell expression property and applying a second reagent as a marker for a second cell expression property.

19. The method of claim 18, wherein the step of applying the cytological detection cocktail includes applying the first and second reagents together to the clusters of cells.

20. The method of claim 8, wherein the step of analyzing the clusters of cells comprising scanning the clusters of cells and measuring an intensity of individual signals detected from the assay and determining how a ratio of the signals detected vary across the clusters of cells.

21. A method for detecting abnormal tissue, the method comprising:
(a) collecting a cell sample with a collector, the cell sample including cell clusters from a local area of a tissue where a potential imbalance of two or more cell expression properties may be exhibited in a state of dysplasia;
(b) transferring at least some of the collected cell clusters to a receiving structure;
(c) applying a cytological detection cocktail to the cell clusters transferred to the receiving structure; and
(d) analyzing the transferred cell clusters for an imbalance of two or more cell expression properties.

22. The method of claim 21, wherein the step of applying a cytological detection cocktail comprises applying a first reagent as a marker for a first cell expression property and applying a second reagent as a marker for a second cell expression property.

23. The method of claim 22, wherein the step of applying a cytological detection cocktail includes applying the first and second reagents together to the transferred cells of a single cell sample.

24. The method of claim 21, wherein the analyzing step further comprising scanning the clusters of cells with a scanning device.

25. A method of screening cell clusters to indicate the existence of cells that are part of a local area where a pre-neoplastic or neoplastic lesion may be present, comprising:
interrogating the cell clusters for two or more biological markers that do not normally occur in the same cell during normal cellular growth, development and function.
